SEQUENCE LISTING (185 sequences)

SEQ ID NO: 1 - RNA, 40 nt, Artificial Sequence
  Artificial, non-naturally occuring RNA aptamers which bind with selectivity/specificity to biliverdin
aaagcaacaaaugucuucgauggcgugggcgcacgguugg

SEQ ID NO: 2 - RNA, 40 nt, Artificial Sequence
  Artificial, non-naturally occuring RNA aptamers which bind with selectivity/specificity to biliverdin
aaauucggaccggugagauaauguucuugcgcugucuacc

SEQ ID NO: 3 - RNA, 40 nt, Artificial Sequence
  Artificial, non-naturally occuring RNA aptamers which bind with selectivity/specificity to biliverdin
aaccauguucuccuuggugaguugugucugcuguguugug

SEQ ID NO: 4 - RNA, 40 nt, Artificial Sequence
  Artificial, non-naturally occuring RNA aptamers which bind with selectivity/specificity to biliverdin
aaccugucguggguuaagagauguggguucgucgucggga

SEQ ID NO: 5 - RNA, 40 nt, Artificial Sequence
  Artificial, non-naturally occuring RNA aptamers which bind with selectivity/specificity to biliverdin
aacuuggggauuuguucaguuggaccgcuugcuuuugagc

SEQ ID NO: 6 - RNA, 40 nt, Artificial Sequence
  Artificial, non-naturally occuring RNA aptamers which bind with selectivity/specificity to biliverdin
aagcugagaugucgagauuaacuuugguaucauuggugau

SEQ ID NO: 7 - RNA, 40 nt, Artificial Sequence
  Artificial, non-naturally occuring RNA aptamers which bind with selectivity/specificity to biliverdin
aaggcgucaucuacugcucuggacuguaaaugccggcugc

SEQ ID NO: 8 - RNA, 40 nt, Artificial Sequence
  Artificial, non-naturally occuring RNA aptamers which bind with selectivity/specificity to biliverdin
aaugaggguggagguuugucuugaccaauugggggcugua

SEQ ID NO: 9 - RNA, 40 nt, Artificial Sequence
  Artificial, non-naturally occuring RNA aptamers which bind with selectivity/specificity to biliverdin
acaacggcaguuccuucgggugaaguuguuuaagugggca

SEQ ID NO: 10 - RNA, 40 nt, Artificial Sequence
  Artificial, non-naturally occuring RNA aptamers which bind with selectivity/specificity to biliverdin
accaugugucacuuugcuccuaaggccuauuuugggacgu

SEQ ID NO: 11 - RNA, 40 nt, Artificial Sequence
  Artificial, non-naturally occuring RNA aptamers which bind with selectivity/specificity to biliverdin
acccgagugagucgguucggcaaauugguuucagugguga

SEQ ID NO: 12 - RNA, 40 nt, Artificial Sequence
  Artificial, non-naturally occuring RNA aptamers which bind with selectivity/specificity to biliverdin
accggaaguguggguacgugucgauucagugguacgggcu

SEQ ID NO: 13 - RNA, 40 nt, Artificial Sequence
  Artificial, non-naturally occuring RNA aptamers which bind with selectivity/specificity to biliverdin
acggcucgaggucgucgggcaugggcuucgugucgcucuc

SEQ ID NO: 14 - RNA, 40 nt, Artificial Sequence
  Artificial, non-naturally occuring RNA aptamers which bind with selectivity/specificity to biliverdin
acgguuauuucuuccuggagaaucacccuaagggauugcg

SEQ ID NO: 15 - RNA, 40 nt, Artificial Sequence
  Artificial, non-naturally occuring RNA aptamers which bind with selectivity/specificity to biliverdin
acgugcacggacagccuagauggacggaaaugguugagug

SEQ ID NO: 16 - RNA, 40 nt, Artificial Sequence
  Artificial, non-naturally occuring RNA aptamers which bind with selectivity/specificity to biliverdin
acuacugucgcgcgucagugucguuguccucacgcucucg

SEQ ID NO: 17 - RNA, 40 nt, Artificial Sequence
  Artificial, non-naturally occuring RNA aptamers which bind with selectivity/specificity to biliverdin
acuuuggggcuucggcggauauuggugucucggugauggc

SEQ ID NO: 18 - RNA, 40 nt, Artificial Sequence
  Artificial, non-naturally occuring RNA aptamers which bind with selectivity/specificity to biliverdin
aggaugaugccucugaugcggaaaucgccauuugcgggaa

SEQ ID NO: 19 - RNA, 40 nt, Artificial Sequence
  Artificial, non-naturally occuring RNA aptamers which bind with selectivity/specificity to biliverdin
aggcaugugugucggcugcaugaaugucucgggugaguug

SEQ ID NO: 20 - RNA, 40 nt, Artificial Sequence
  Artificial, non-naturally occuring RNA aptamers which bind with selectivity/specificity to biliverdin
agggcaauucuuacucucucaggugggcguugaugucgug

SEQ ID NO: 21 - RNA, 40 nt, Artificial Sequence
  Artificial, non-naturally occuring RNA aptamers which bind with selectivity/specificity to biliverdin
aucccgguagugagcagagugugucuugaucgaucuauug

SEQ ID NO: 22 - RNA, 40 nt, Artificial Sequence
  Artificial, non-naturally occuring RNA aptamers which bind with selectivity/specificity to biliverdin
auggaaaucugguguugucagaaaucggaucacuuagugg

SEQ ID NO: 23 - RNA, 40 nt, Artificial Sequence
  Artificial, non-naturally occuring RNA aptamers which bind with selectivity/specificity to biliverdin
auggcucuugccgaugauaguuguuccggaacggcuaggg

SEQ ID NO: 24 - RNA, 40 nt, Artificial Sequence
  Artificial, non-naturally occuring RNA aptamers which bind with selectivity/specificity to biliverdin
auggguggaaaacuguuacgggagcuaauaagucggaacc

SEQ ID NO: 25 - RNA, 40 nt, Artificial Sequence
  Artificial, non-naturally occuring RNA aptamers which bind with selectivity/specificity to biliverdin
augucuacccaaccugcaaauuggcgagugguguugcgug

SEQ ID NO: 26 - RNA, 40 nt, Artificial Sequence
  Artificial, non-naturally occuring RNA aptamers which bind with selectivity/specificity to biliverdin
caaaguggaagugucccccaauucugucgcuguuguugca

SEQ ID NO: 27 - RNA, 40 nt, Artificial Sequence
  Artificial, non-naturally occuring RNA aptamers which bind with selectivity/specificity to biliverdin
caaggaguucaagcgauucuuguauuucgagggaugaggc

SEQ ID NO: 28 - RNA, 40 nt, Artificial Sequence
  Artificial, non-naturally occuring RNA aptamers which bind with selectivity/specificity to biliverdin
caaugucuuuccaaggagaucugggugggacgaggucggc

SEQ ID NO: 29 - RNA, 40 nt, Artificial Sequence
  Artificial, non-naturally occuring RNA aptamers which bind with selectivity/specificity to biliverdin
cacacuuuugcggugaguguagucgcuccgagugaccugg

SEQ ID NO: 30 - RNA, 40 nt, Artificial Sequence
  Artificial, non-naturally occuring RNA aptamers which bind with selectivity/specificity to biliverdin
cacugugcggucacguucuuguaguuuggucuauugcguc

SEQ ID NO: 31 - RNA, 40 nt, Artificial Sequence
  Artificial, non-naturally occuring RNA aptamers which bind with selectivity/specificity to biliverdin
cacuucggaagauacguguuuagcagagugugauagggcu

SEQ ID NO: 32 - RNA, 40 nt, Artificial Sequence
  Artificial, non-naturally occuring RNA aptamers which bind with selectivity/specificity to biliverdin
cagcuuuccugacuucucgucaacgaugagucgacuuggg

SEQ ID NO: 33 - RNA, 40 nt, Artificial Sequence
  Artificial, non-naturally occuring RNA aptamers which bind with selectivity/specificity to biliverdin
cagucggaucgaacacggcaaaagagcgugucggagaaac

SEQ ID NO: 34 - RNA, 40 nt, Artificial Sequence
  Artificial, non-naturally occuring RNA aptamers which bind with selectivity/specificity to biliverdin
cauccagacgugguucuuguucggaacguguaugaauggu

SEQ ID NO: 35 - RNA, 40 nt, Artificial Sequence
  Artificial, non-naturally occuring RNA aptamers which bind with selectivity/specificity to biliverdin
ccaacaauuggucuggugucgaucagguguagcaugcucg

SEQ ID NO: 36 - RNA, 40 nt, Artificial Sequence
  Artificial, non-naturally occuring RNA aptamers which bind with selectivity/specificity to biliverdin
ccaggugcauccgccgaguugucuguuugcuuaggugagg

SEQ ID NO: 37 - RNA, 40 nt, Artificial Sequence
  Artificial, non-naturally occuring RNA aptamers which bind with selectivity/specificity to biliverdin
ccaugcgcucgauagaagauugaggggccggaggaagcgg

SEQ ID NO: 38 - RNA, 40 nt, Artificial Sequence
  Artificial, non-naturally occuring RNA aptamers which bind with selectivity/specificity to biliverdin
ccaugcuggggaaaguggauggccuuguucuccgcggcuc

SEQ ID NO: 39 - RNA, 40 nt, Artificial Sequence
  Artificial, non-naturally occuring RNA aptamers which bind with selectivity/specificity to biliverdin
ccgaugacacuguugcgucuccagcaccaggcaagccgag

SEQ ID NO: 40 - RNA, 40 nt, Artificial Sequence
  Artificial, non-naturally occuring RNA aptamers which bind with selectivity/specificity to biliverdin
ccggaggugaaugagcaggccuuccaaacgucaggcagcg

SEQ ID NO: 41 - RNA, 40 nt, Artificial Sequence
  Artificial, non-naturally occuring RNA aptamers which bind with selectivity/specificity to biliverdin
ccggccuugggagugauugucgcucgguaagauguuagug

SEQ ID NO: 42 - RNA, 40 nt, Artificial Sequence
  Artificial, non-naturally occuring RNA aptamers which bind with selectivity/specificity to biliverdin
ccgguuugcugcucuuggguaggacuucaaugcguugaug

SEQ ID NO: 43 - RNA, 40 nt, Artificial Sequence
  Artificial, non-naturally occuring RNA aptamers which bind with selectivity/specificity to biliverdin
ccguaaagagaggagcgggguuucaagucgcgugagcgag

SEQ ID NO: 44 - RNA, 40 nt, Artificial Sequence
  Artificial, non-naturally occuring RNA aptamers which bind with selectivity/specificity to biliverdin
ccguagccaaguguagugauuguagugcaagccacucgca

SEQ ID NO: 45 - RNA, 40 nt, Artificial Sequence
  Artificial, non-naturally occuring RNA aptamers which bind with selectivity/specificity to biliverdin
ccgugagguuaaugcucuaugcgcgaguuuaacggagggc

SEQ ID NO: 46 - RNA, 40 nt, Artificial Sequence
  Artificial, non-naturally occuring RNA aptamers which bind with selectivity/specificity to biliverdin
ccgugugucaauguuggaaccugaguuggaaacggucacu

SEQ ID NO: 47 - RNA, 40 nt, Artificial Sequence
  Artificial, non-naturally occuring RNA aptamers which bind with selectivity/specificity to biliverdin
ccguugaccaguuaagguaguccccaucgagguuccgugu

SEQ ID NO: 48 - RNA, 40 nt, Artificial Sequence
  Artificial, non-naturally occuring RNA aptamers which bind with selectivity/specificity to biliverdin
ccguuggacggacgagugacuuugugcuucuggcaguugu

SEQ ID NO: 49 - RNA, 40 nt, Artificial Sequence
  Artificial, non-naturally occuring RNA aptamers which bind with selectivity/specificity to biliverdin
ccucggugcugaguaauuaugucuggucuucgagaauggg

SEQ ID NO: 50 - RNA, 40 nt, Artificial Sequence
  Artificial, non-naturally occuring RNA aptamers which bind with selectivity/specificity to biliverdin
ccugggcagaauuuguaguguuccaagugggugucuguga

SEQ ID NO: 51 - RNA, 40 nt, Artificial Sequence
  Artificial, non-naturally occuring RNA aptamers which bind with selectivity/specificity to biliverdin
cgaacgaaauggcugcugcaguuauuagaaucgagagucg

SEQ ID NO: 52 - RNA, 40 nt, Artificial Sequence
  Artificial, non-naturally occuring RNA aptamers which bind with selectivity/specificity to biliverdin
cgacccgugcagcuauucuuggaagaauuguucggggcuu

SEQ ID NO: 53 - RNA, 40 nt, Artificial Sequence
  Artificial, non-naturally occuring RNA aptamers which bind with selectivity/specificity to biliverdin
cgacgagugaucgugggagcgggauagcugaaggacgcca

SEQ ID NO: 54 - RNA, 40 nt, Artificial Sequence
  Artificial, non-naturally occuring RNA aptamers which bind with selectivity/specificity to biliverdin
cgaucgucguucauguuugcuuccauggaaaagccaaugc

SEQ ID NO: 55 - RNA, 40 nt, Artificial Sequence
  Artificial, non-naturally occuring RNA aptamers which bind with selectivity/specificity to biliverdin
cgaugguguucaaaguuggacgagacacacugagccagga

SEQ ID NO: 56 - RNA, 40 nt, Artificial Sequence
  Artificial, non-naturally occuring RNA aptamers which bind with selectivity/specificity to biliverdin
cgauguuuggggugaauccuugaauucgugcugugugucc

SEQ ID NO: 57 - RNA, 40 nt, Artificial Sequence
  Artificial, non-naturally occuring RNA aptamers which bind with selectivity/specificity to biliverdin
cggaagugacggcguugcucucggcugagugaucugacua

SEQ ID NO: 58 - RNA, 40 nt, Artificial Sequence
  Artificial, non-naturally occuring RNA aptamers which bind with selectivity/specificity to biliverdin
cggaauguauagucagagcauccugauucugcuagcagug

SEQ ID NO: 59 - RNA, 40 nt, Artificial Sequence
  Artificial, non-naturally occuring RNA aptamers which bind with selectivity/specificity to biliverdin
cggccgugcgcauguggaggggugaguucuuguuggagac

SEQ ID NO: 60 - RNA, 40 nt, Artificial Sequence
  Artificial, non-naturally occuring RNA aptamers which bind with selectivity/specificity to biliverdin
cguacuguucgugugucugcgugaucgauaccuuccgcua

SEQ ID NO: 61 - RNA, 40 nt, Artificial Sequence
  Artificial, non-naturally occuring RNA aptamers which bind with selectivity/specificity to biliverdin
cguggauuaggcggaggucuucuaguuaguugugugguuc

SEQ ID NO: 62 - RNA, 40 nt, Artificial Sequence
  Artificial, non-naturally occuring RNA aptamers which bind with selectivity/specificity to biliverdin
cguguuuguucgucucaguguugcuucgcgguacguguag

SEQ ID NO: 63 - RNA, 40 nt, Artificial Sequence
  Artificial, non-naturally occuring RNA aptamers which bind with selectivity/specificity to biliverdin
cguugcguggaagaggucucucguguggccgucugaggga

SEQ ID NO: 64 - RNA, 40 nt, Artificial Sequence
  Artificial, non-naturally occuring RNA aptamers which bind with selectivity/specificity to biliverdin
cuacagaaaccguuucgguugguugguaggggagaguugg

SEQ ID NO: 65 - RNA, 40 nt, Artificial Sequence
  Artificial, non-naturally occuring RNA aptamers which bind with selectivity/specificity to biliverdin
cuacgucuuggcucuauggugaacguccuggcagugcugg

SEQ ID NO: 66 - RNA, 40 nt, Artificial Sequence
  Artificial, non-naturally occuring RNA aptamers which bind with selectivity/specificity to biliverdin
cuccugaagcgcuuguucuugacacugagugugguggggu

SEQ ID NO: 67 - RNA, 40 nt, Artificial Sequence
  Artificial, non-naturally occuring RNA aptamers which bind with selectivity/specificity to biliverdin
cucgggccuugagauacacgcguucgaacugcaaguguac

SEQ ID NO: 68 - RNA, 40 nt, Artificial Sequence
  Artificial, non-naturally occuring RNA aptamers which bind with selectivity/specificity to biliverdin
cucgugguucaaaagucgaugcauugauucuguaucugcc

SEQ ID NO: 69 - RNA, 40 nt, Artificial Sequence
  Artificial, non-naturally occuring RNA aptamers which bind with selectivity/specificity to biliverdin
cucucugaauguggacucuuggguuuagcuggaagugcga

SEQ ID NO: 70 - RNA, 40 nt, Artificial Sequence
  Artificial, non-naturally occuring RNA aptamers which bind with selectivity/specificity to biliverdin
cucugcaagagaaucgagugcugauuacccagggaguuac

SEQ ID NO: 71 - RNA, 40 nt, Artificial Sequence
  Artificial, non-naturally occuring RNA aptamers which bind with selectivity/specificity to biliverdin
cugagaaugagggucgaaucuugagucgaugaggaacggu

SEQ ID NO: 72 - RNA, 40 nt, Artificial Sequence
  Artificial, non-naturally occuring RNA aptamers which bind with selectivity/specificity to biliverdin
cuggaugugguuagcuuggauuuggucguauuggaaacgg

SEQ ID NO: 73 - RNA, 40 nt, Artificial Sequence
  Artificial, non-naturally occuring RNA aptamers which bind with selectivity/specificity to biliverdin
cuuccagggcuugaugcgaagccuguaaagacugaagcug

SEQ ID NO: 74 - RNA, 40 nt, Artificial Sequence
  Artificial, non-naturally occuring RNA aptamers which bind with selectivity/specificity to biliverdin
cuuccugauuuccuucugagaugguugcgacuuucuggcg

SEQ ID NO: 75 - RNA, 40 nt, Artificial Sequence
  Artificial, non-naturally occuring RNA aptamers which bind with selectivity/specificity to biliverdin
cuugggcgggaaauucggugcucgcaagggucaacgcggu

SEQ ID NO: 76 - RNA, 40 nt, Artificial Sequence
  Artificial, non-naturally occuring RNA aptamers which bind with selectivity/specificity to biliverdin
cuugggugauggguuuuggagacuuucgcuugguuuggag

SEQ ID NO: 77 - RNA, 40 nt, Artificial Sequence
  Artificial, non-naturally occuring RNA aptamers which bind with selectivity/specificity to biliverdin
gaagguuggaaugagcggauggcuucguuugucguucagg

SEQ ID NO: 78 - RNA, 40 nt, Artificial Sequence
  Artificial, non-naturally occuring RNA aptamers which bind with selectivity/specificity to biliverdin
gaaucgccuacgugacguguauucuuaggaucucuauggc

SEQ ID NO: 79 - RNA, 40 nt, Artificial Sequence
  Artificial, non-naturally occuring RNA aptamers which bind with selectivity/specificity to biliverdin
gaaugguguguggauugaugcguucugucgcugggcgaag

SEQ ID NO: 80 - RNA, 40 nt, Artificial Sequence
  Artificial, non-naturally occuring RNA aptamers which bind with selectivity/specificity to biliverdin
gagaauuuaugucgggucuucgguacgagucugccacugc

SEQ ID NO: 81 - RNA, 40 nt, Artificial Sequence
  Artificial, non-naturally occuring RNA aptamers which bind with selectivity/specificity to biliverdin
gagagaaauucuaucgcgucgcuuuguaccugagucgugu

SEQ ID NO: 82 - RNA, 40 nt, Artificial Sequence
  Artificial, non-naturally occuring RNA aptamers which bind with selectivity/specificity to biliverdin
gagcacagaagcaaccgggaggcagaagccacgcggcgga

SEQ ID NO: 83 - RNA, 40 nt, Artificial Sequence
  Artificial, non-naturally occuring RNA aptamers which bind with selectivity/specificity to biliverdin
gagggaguagaguucguucuuguugaaugcgacugggauc

SEQ ID NO: 84 - RNA, 40 nt, Artificial Sequence
  Artificial, non-naturally occuring RNA aptamers which bind with selectivity/specificity to biliverdin
gaugaacguuguucuaugucuggugauugagagcuaucug

SEQ ID NO: 85 - RNA, 40 nt, Artificial Sequence
  Artificial, non-naturally occuring RNA aptamers which bind with selectivity/specificity to biliverdin
gauuggguggauuucggugcucggcuggccuguacgaucg

SEQ ID NO: 86 - RNA, 40 nt, Artificial Sequence
  Artificial, non-naturally occuring RNA aptamers which bind with selectivity/specificity to biliverdin
gcauccuuguuucagcguuuucguucguugacuauggugg

SEQ ID NO: 87 - RNA, 40 nt, Artificial Sequence
  Artificial, non-naturally occuring RNA aptamers which bind with selectivity/specificity to biliverdin
gccacgucggacuaguugcuguguuuucgugcgcuuuuga

SEQ ID NO: 88 - RNA, 40 nt, Artificial Sequence
  Artificial, non-naturally occuring RNA aptamers which bind with selectivity/specificity to biliverdin
gccauugaguauagauuguugaauguuggcguagucgaug

SEQ ID NO: 89 - RNA, 40 nt, Artificial Sequence
  Artificial, non-naturally occuring RNA aptamers which bind with selectivity/specificity to biliverdin
gccggcauccgaccugugcgucaaagucgggaucgcgcga

SEQ ID NO: 90 - RNA, 40 nt, Artificial Sequence
  Artificial, non-naturally occuring RNA aptamers which bind with selectivity/specificity to biliverdin
gccugcacuagcuugugaauuucgagucgagacguaggcg

SEQ ID NO: 91 - RNA, 40 nt, Artificial Sequence
  Artificial, non-naturally occuring RNA aptamers which bind with selectivity/specificity to biliverdin
gccugugcgaccaguugugacaacggguacauugggugcg

SEQ ID NO: 92 - RNA, 40 nt, Artificial Sequence
  Artificial, non-naturally occuring RNA aptamers which bind with selectivity/specificity to biliverdin
gccuuggaauccacaugugaguugucugaucuucaucgug

SEQ ID NO: 93 - RNA, 40 nt, Artificial Sequence
  Artificial, non-naturally occuring RNA aptamers which bind with selectivity/specificity to biliverdin
ggaagcccgcgugggauuccagaaggcgcucugucgguga

SEQ ID NO: 94 - RNA, 40 nt, Artificial Sequence
  Artificial, non-naturally occuring RNA aptamers which bind with selectivity/specificity to biliverdin
ggagggcgcuucuugccaggaaaaaugucugggcugugcu

SEQ ID NO: 95 - RNA, 40 nt, Artificial Sequence
  Artificial, non-naturally occuring RNA aptamers which bind with selectivity/specificity to biliverdin
ggaggguucucuugacguccuaaggugcggcugguccagg

SEQ ID NO: 96 - RNA, 40 nt, Artificial Sequence
  Artificial, non-naturally occuring RNA aptamers which bind with selectivity/specificity to biliverdin
ggagucaugcggagauggaaucuugggugugugggcuaac

SEQ ID NO: 97 - RNA, 40 nt, Artificial Sequence
  Artificial, non-naturally occuring RNA aptamers which bind with selectivity/specificity to biliverdin
ggagucuguuugcgaaguuccaauauagcuauuggaguga

SEQ ID NO: 98 - RNA, 40 nt, Artificial Sequence
  Artificial, non-naturally occuring RNA aptamers which bind with selectivity/specificity to biliverdin
ggaguguauguucaauguguccgggcugcggucgugguag

SEQ ID NO: 99 - RNA, 40 nt, Artificial Sequence
  Artificial, non-naturally occuring RNA aptamers which bind with selectivity/specificity to biliverdin
ggauugugcuuccgucugguugaugagguugcuguggucg

SEQ ID NO: 100 - RNA, 40 nt, Artificial Sequence
  Artificial, non-naturally occuring RNA aptamers which bind with selectivity/specificity to biliverdin
ggcaccgcuguggggcuguucgucuucucgagaggugagg

SEQ ID NO: 101 - RNA, 40 nt, Artificial Sequence
  Artificial, non-naturally occuring RNA aptamers which bind with selectivity/specificity to biliverdin
gggcaucacgggcgaaucgacggagaaguuuguggggagg

SEQ ID NO: 102 - RNA, 40 nt, Artificial Sequence
  Artificial, non-naturally occuring RNA aptamers which bind with selectivity/specificity to biliverdin
ggggggucgucucugugauuguggagcucaaaacauguca

SEQ ID NO: 103 - RNA, 40 nt, Artificial Sequence
  Artificial, non-naturally occuring RNA aptamers which bind with selectivity/specificity to biliverdin
ggguggugcucuggaacgggguuucgguucaagcugcggu

SEQ ID NO: 104 - RNA, 40 nt, Artificial Sequence
  Artificial, non-naturally occuring RNA aptamers which bind with selectivity/specificity to biliverdin
ggguuacugugauucgugaugcuguuaucuuggagucugu

SEQ ID NO: 105 - RNA, 40 nt, Artificial Sequence
  Artificial, non-naturally occuring RNA aptamers which bind with selectivity/specificity to biliverdin
gucaccggguaucugugguugaucgaugguaauuggugcg

SEQ ID NO: 106 - RNA, 40 nt, Artificial Sequence
  Artificial, non-naturally occuring RNA aptamers which bind with selectivity/specificity to biliverdin
gucgaucagaccgucucuccaccugcgacgaggcuggaug

SEQ ID NO: 107 - RNA, 40 nt, Artificial Sequence
  Artificial, non-naturally occuring RNA aptamers which bind with selectivity/specificity to biliverdin
gucgccuagugaguuucguaggcagauucuauccaggguc

SEQ ID NO: 108 - RNA, 40 nt, Artificial Sequence
  Artificial, non-naturally occuring RNA aptamers which bind with selectivity/specificity to biliverdin
gucggagaucugaaggucuggaaacucugcugucgcgcug

SEQ ID NO: 109 - RNA, 40 nt, Artificial Sequence
  Artificial, non-naturally occuring RNA aptamers which bind with selectivity/specificity to biliverdin
gucugagaagugagaugaacauucucggaguccgacggug

SEQ ID NO: 110 - RNA, 40 nt, Artificial Sequence
  Artificial, non-naturally occuring RNA aptamers which bind with selectivity/specificity to biliverdin
gucuggucuguggguuauugauucuuucuaagcuucgauc

SEQ ID NO: 111 - RNA, 40 nt, Artificial Sequence
  Artificial, non-naturally occuring RNA aptamers which bind with selectivity/specificity to biliverdin
gucuucaaggagaagaggcucgguuaaaaccaucuucggg

SEQ ID NO: 112 - RNA, 40 nt, Artificial Sequence
  Artificial, non-naturally occuring RNA aptamers which bind with selectivity/specificity to biliverdin
gucuucucgagcgcguacucucgguugaugacuccaucga

SEQ ID NO: 113 - RNA, 40 nt, Artificial Sequence
  Artificial, non-naturally occuring RNA aptamers which bind with selectivity/specificity to biliverdin
gugaggggauagucugggucaccagugaguaugugguucg

SEQ ID NO: 114 - RNA, 40 nt, Artificial Sequence
  Artificial, non-naturally occuring RNA aptamers which bind with selectivity/specificity to biliverdin
gugguguuuaucuucgaagccggcuggugcgcgucacggu

SEQ ID NO: 115 - RNA, 40 nt, Artificial Sequence
  Artificial, non-naturally occuring RNA aptamers which bind with selectivity/specificity to biliverdin
guguuggaggacagggcguaugauucugacgucggggauc

SEQ ID NO: 116 - RNA, 40 nt, Artificial Sequence
  Artificial, non-naturally occuring RNA aptamers which bind with selectivity/specificity to biliverdin
guuaccguugucggucaucuucguugaaggucacguggcg

SEQ ID NO: 117 - RNA, 40 nt, Artificial Sequence
  Artificial, non-naturally occuring RNA aptamers which bind with selectivity/specificity to biliverdin
guuaugugccacuacgcucggagacauucgcugaguguga

SEQ ID NO: 118 - RNA, 40 nt, Artificial Sequence
  Artificial, non-naturally occuring RNA aptamers which bind with selectivity/specificity to biliverdin
guugggauggucuguugaugcacgcaguaagcacucgugg

SEQ ID NO: 119 - RNA, 40 nt, Artificial Sequence
  Artificial, non-naturally occuring RNA aptamers which bind with selectivity/specificity to biliverdin
uaaucggauuuugggugguguuugaaugucaggaguagcg

SEQ ID NO: 120 - RNA, 40 nt, Artificial Sequence
  Artificial, non-naturally occuring RNA aptamers which bind with selectivity/specificity to biliverdin
uacugggauaaguugguggcggguucuuucuggcugaggc

SEQ ID NO: 121 - RNA, 40 nt, Artificial Sequence
  Artificial, non-naturally occuring RNA aptamers which bind with selectivity/specificity to biliverdin
uagcucaugucguauacuguacuccaaggguugcguugac

SEQ ID NO: 122 - RNA, 40 nt, Artificial Sequence
  Artificial, non-naturally occuring RNA aptamers which bind with selectivity/specificity to biliverdin
uaguggguuuugcguugguucgggccccuaucgcgggaag

SEQ ID NO: 123 - RNA, 40 nt, Artificial Sequence
  Artificial, non-naturally occuring RNA aptamers which bind with selectivity/specificity to biliverdin
ucaacacagugcuguuuauaugggugauuguucguaccug

SEQ ID NO: 124 - RNA, 40 nt, Artificial Sequence
  Artificial, non-naturally occuring RNA aptamers which bind with selectivity/specificity to biliverdin
ucacgcgauugaugguuuggguucucugcugggauugggu

SEQ ID NO: 125 - RNA, 40 nt, Artificial Sequence
  Artificial, non-naturally occuring RNA aptamers which bind with selectivity/specificity to biliverdin
ucagaaagaacaucgcguauuuccguuuuccgacguccug

SEQ ID NO: 126 - RNA, 40 nt, Artificial Sequence
  Artificial, non-naturally occuring RNA aptamers which bind with selectivity/specificity to biliverdin
ucagaccuuuacgugagugauggauacuuuugagugggag

SEQ ID NO: 127 - RNA, 40 nt, Artificial Sequence
  Artificial, non-naturally occuring RNA aptamers which bind with selectivity/specificity to biliverdin
uccagggcuguccuuaauuaagggaguugaggugucggcg

SEQ ID NO: 128 - RNA, 40 nt, Artificial Sequence
  Artificial, non-naturally occuring RNA aptamers which bind with selectivity/specificity to biliverdin
uccaguggacgcuugagguagcggugaguguggggccaau

SEQ ID NO: 129 - RNA, 40 nt, Artificial Sequence
  Artificial, non-naturally occuring RNA aptamers which bind with selectivity/specificity to biliverdin
ucccggaauagggacggguggacacuuggagaacugggcu

SEQ ID NO: 130 - RNA, 40 nt, Artificial Sequence
  Artificial, non-naturally occuring RNA aptamers which bind with selectivity/specificity to biliverdin
uccgagugcguugugucugcuguacguaauggguuccuca

SEQ ID NO: 131 - RNA, 40 nt, Artificial Sequence
  Artificial, non-naturally occuring RNA aptamers which bind with selectivity/specificity to biliverdin
uccgaguucguggucgauuguguccagcccugagcaaaug

SEQ ID NO: 132 - RNA, 40 nt, Artificial Sequence
  Artificial, non-naturally occuring RNA aptamers which bind with selectivity/specificity to biliverdin
uccucaguccgaccugguguagucaagugggguuacagug

SEQ ID NO: 133 - RNA, 40 nt, Artificial Sequence
  Artificial, non-naturally occuring RNA aptamers which bind with selectivity/specificity to biliverdin
uccuccggccuuggucggugcagagucggaacuaugcaug

SEQ ID NO: 134 - RNA, 40 nt, Artificial Sequence
  Artificial, non-naturally occuring RNA aptamers which bind with selectivity/specificity to biliverdin
uccucgguuuagcaguacucguguuucggucgugaaggau

SEQ ID NO: 135 - RNA, 40 nt, Artificial Sequence
  Artificial, non-naturally occuring RNA aptamers which bind with selectivity/specificity to biliverdin
uccugagaaguccuugugaguguuggccaaauucgugcug

SEQ ID NO: 136 - RNA, 40 nt, Artificial Sequence
  Artificial, non-naturally occuring RNA aptamers which bind with selectivity/specificity to biliverdin
uccuguuccaauguggacggcucggggcaauugaggaggc

SEQ ID NO: 137 - RNA, 40 nt, Artificial Sequence
  Artificial, non-naturally occuring RNA aptamers which bind with selectivity/specificity to biliverdin
ucgagaaggguguacuccgugcgugugcccgugcuguuag

SEQ ID NO: 138 - RNA, 40 nt, Artificial Sequence
  Artificial, non-naturally occuring RNA aptamers which bind with selectivity/specificity to biliverdin
ucgagguuuuucuggguuagucugcugcgugcgaucugug

SEQ ID NO: 139 - RNA, 40 nt, Artificial Sequence
  Artificial, non-naturally occuring RNA aptamers which bind with selectivity/specificity to biliverdin
ucgaugguucggcauucucuacuccucgucaagugcaucc

SEQ ID NO: 140 - RNA, 40 nt, Artificial Sequence
  Artificial, non-naturally occuring RNA aptamers which bind with selectivity/specificity to biliverdin
ucggaauggugcgggugaucuaaucguugugaggaguuug

SEQ ID NO: 141 - RNA, 40 nt, Artificial Sequence
  Artificial, non-naturally occuring RNA aptamers which bind with selectivity/specificity to biliverdin
ucggccuugagauacucagcucugaaagucguugcugacc

SEQ ID NO: 142 - RNA, 40 nt, Artificial Sequence
  Artificial, non-naturally occuring RNA aptamers which bind with selectivity/specificity to biliverdin
ucgggaguacgugucuacuaaaggaguccucaguucggag

SEQ ID NO: 143 - RNA, 40 nt, Artificial Sequence
  Artificial, non-naturally occuring RNA aptamers which bind with selectivity/specificity to biliverdin
ucggguugugcugagucuugacugauuggcugcuggcaga

SEQ ID NO: 144 - RNA, 40 nt, Artificial Sequence
  Artificial, non-naturally occuring RNA aptamers which bind with selectivity/specificity to biliverdin
ucgguguugcgaauauuguugguuuuccuucuguggcacc

SEQ ID NO: 145 - RNA, 40 nt, Artificial Sequence
  Artificial, non-naturally occuring RNA aptamers which bind with selectivity/specificity to biliverdin
ucgugaaggucguggugugagagccaugcacgugccgcaa

SEQ ID NO: 146 - RNA, 40 nt, Artificial Sequence
  Artificial, non-naturally occuring RNA aptamers which bind with selectivity/specificity to biliverdin
ucgugauuguucggcaccaucuuucucguggugaguaugc

SEQ ID NO: 147 - RNA, 40 nt, Artificial Sequence
  Artificial, non-naturally occuring RNA aptamers which bind with selectivity/specificity to biliverdin
ucguugcagugaaagucguugucgugccauguaccgcagg

SEQ ID NO: 148 - RNA, 40 nt, Artificial Sequence
  Artificial, non-naturally occuring RNA aptamers which bind with selectivity/specificity to biliverdin
ucguuucggugaaccacugggucggcucauucgugacgga

SEQ ID NO: 149 - RNA, 40 nt, Artificial Sequence
  Artificial, non-naturally occuring RNA aptamers which bind with selectivity/specificity to biliverdin
ucucaggcugagucucucgcgaaacgggaccgaguguauc

SEQ ID NO: 150 - RNA, 40 nt, Artificial Sequence
  Artificial, non-naturally occuring RNA aptamers which bind with selectivity/specificity to biliverdin
ugaaagcacgacuuucggggcacagacggacuccaguggg

SEQ ID NO: 151 - RNA, 40 nt, Artificial Sequence
  Artificial, non-naturally occuring RNA aptamers which bind with selectivity/specificity to biliverdin
ugacguaagcgaagguucgguuggcucuagugcuucgugg

SEQ ID NO: 152 - RNA, 40 nt, Artificial Sequence
  Artificial, non-naturally occuring RNA aptamers which bind with selectivity/specificity to biliverdin
ugagaggcauucuugauuucgcuaugaagacuugagcuug

SEQ ID NO: 153 - RNA, 40 nt, Artificial Sequence
  Artificial, non-naturally occuring RNA aptamers which bind with selectivity/specificity to biliverdin
ugaugugauggcucaugaggaucacaauucguugcacggg

SEQ ID NO: 154 - RNA, 40 nt, Artificial Sequence
  Artificial, non-naturally occuring RNA aptamers which bind with selectivity/specificity to biliverdin
ugauuugagauuguuggagacuugguuugguuguggugac

SEQ ID NO: 155 - RNA, 40 nt, Artificial Sequence
  Artificial, non-naturally occuring RNA aptamers which bind with selectivity/specificity to biliverdin
ugcagcaagucagaagagcucuaagacagcgugcggugcg

SEQ ID NO: 156 - RNA, 40 nt, Artificial Sequence
  Artificial, non-naturally occuring RNA aptamers which bind with selectivity/specificity to biliverdin
ugcccuugugggaauucggauuuguucuugcuggagagga

SEQ ID NO: 157 - RNA, 40 nt, Artificial Sequence
  Artificial, non-naturally occuring RNA aptamers which bind with selectivity/specificity to biliverdin
ugcugguggcugggaauucggacuccauaaguuguuugcg

SEQ ID NO: 158 - RNA, 40 nt, Artificial Sequence
  Artificial, non-naturally occuring RNA aptamers which bind with selectivity/specificity to biliverdin
uggccgguuucuuggaucgcuagugaguauauccgugacu

SEQ ID NO: 159 - RNA, 40 nt, Artificial Sequence
  Artificial, non-naturally occuring RNA aptamers which bind with selectivity/specificity to biliverdin
uggcuuguugagcagucggucacagucuccggguggcggc

SEQ ID NO: 160 - RNA, 40 nt, Artificial Sequence
  Artificial, non-naturally occuring RNA aptamers which bind with selectivity/specificity to biliverdin
ugggaaacagacguugacgguguuccuuguggcgcguggg

SEQ ID NO: 161 - RNA, 40 nt, Artificial Sequence
  Artificial, non-naturally occuring RNA aptamers which bind with selectivity/specificity to biliverdin
ugggcuguuaugcgaaggucacguugcguucgcgaguacc

SEQ ID NO: 162 - RNA, 40 nt, Artificial Sequence
  Artificial, non-naturally occuring RNA aptamers which bind with selectivity/specificity to biliverdin
uggugaagccacagggguuagaucuuguugaagagcugug

SEQ ID NO: 163 - RNA, 40 nt, Artificial Sequence
  Artificial, non-naturally occuring RNA aptamers which bind with selectivity/specificity to biliverdin
ugguuggcguaaaucgaguaguuucucauguugguggucu

SEQ ID NO: 164 - RNA, 40 nt, Artificial Sequence
  Artificial, non-naturally occuring RNA aptamers which bind with selectivity/specificity to biliverdin
ugguuugccucguuuugacuguacaugugugaagugcgga

SEQ ID NO: 165 - RNA, 40 nt, Artificial Sequence
  Artificial, non-naturally occuring RNA aptamers which bind with selectivity/specificity to biliverdin
uguccuccauccaguggugauucgguguacugucaggugg

SEQ ID NO: 166 - RNA, 40 nt, Artificial Sequence
  Artificial, non-naturally occuring RNA aptamers which bind with selectivity/specificity to biliverdin
ugucgcauagaccgugagcccguucgaggugcgguaacua

SEQ ID NO: 167 - RNA, 40 nt, Artificial Sequence
  Artificial, non-naturally occuring RNA aptamers which bind with selectivity/specificity to biliverdin
ugucucugaaagguuugcugugcguuucuuucggucgguu

SEQ ID NO: 168 - RNA, 40 nt, Artificial Sequence
  Artificial, non-naturally occuring RNA aptamers which bind with selectivity/specificity to biliverdin
ugugaacugggauuguugucuuucuggggugugggagugu

SEQ ID NO: 169 - RNA, 40 nt, Artificial Sequence
  Artificial, non-naturally occuring RNA aptamers which bind with selectivity/specificity to biliverdin
ugugaggcuacuugcagcgucaggcucuguccucggguug

SEQ ID NO: 170 - RNA, 40 nt, Artificial Sequence
  Artificial, non-naturally occuring RNA aptamers which bind with selectivity/specificity to biliverdin
ugugaggguguuguucguucucuggaaucggcuucuugcg

SEQ ID NO: 171 - RNA, 40 nt, Artificial Sequence
  Artificial, non-naturally occuring RNA aptamers which bind with selectivity/specificity to biliverdin
ugugagugaguuugcguuuaggaucucaagugaagugccc

SEQ ID NO: 172 - RNA, 40 nt, Artificial Sequence
  Artificial, non-naturally occuring RNA aptamers which bind with selectivity/specificity to biliverdin
uguuggugcgaguguucaaguuugcuaaggcgugaaccgc

SEQ ID NO: 173 - RNA, 40 nt, Artificial Sequence
  Artificial, non-naturally occuring RNA aptamers which bind with selectivity/specificity to biliverdin
uguuugggugugaugguuguacccgggucguucuugccgu

SEQ ID NO: 174 - RNA, 40 nt, Artificial Sequence
  Artificial, non-naturally occuring RNA aptamers which bind with selectivity/specificity to biliverdin
uucacugagcaauuugcuugagucggaggagagcuuacug

SEQ ID NO: 175 - RNA, 40 nt, Artificial Sequence
  Artificial, non-naturally occuring RNA aptamers which bind with selectivity/specificity to biliverdin
uucgcaauaagugaguauuggacuugugcacucgggugag

SEQ ID NO: 176 - RNA, 40 nt, Artificial Sequence
  Artificial, non-naturally occuring RNA aptamers which bind with selectivity/specificity to biliverdin
uucgggacugagcccaagaaugugaucuucacuucgaucg

SEQ ID NO: 177 - RNA, 40 nt, Artificial Sequence
  Artificial, non-naturally occuring RNA aptamers which bind with selectivity/specificity to biliverdin
uucgugucuugaacacaggugaugcguuuggcugucggaa

SEQ ID NO: 178 - RNA, 40 nt, Artificial Sequence
  Artificial, non-naturally occuring RNA aptamers which bind with selectivity/specificity to biliverdin
uugacgcugaguuguugucugauggagaggagcggaugag

SEQ ID NO: 179 - RNA, 40 nt, Artificial Sequence
  Artificial, non-naturally occuring RNA aptamers which bind with selectivity/specificity to biliverdin
uugagagauguggauguugucgaaggaaaagugagcuggc

SEQ ID NO: 180 - RNA, 40 nt, Artificial Sequence
  Artificial, non-naturally occuring RNA aptamers which bind with selectivity/specificity to biliverdin
uugcccuggauggaccacagcuucaaaagagaggucggag

SEQ ID NO: 181 - RNA, 40 nt, Artificial Sequence
  Artificial, non-naturally occuring RNA aptamers which bind with selectivity/specificity to biliverdin
uuggauugcauuggacuguuauucagcggcucauguggcg

SEQ ID NO: 182 - RNA, 40 nt, Artificial Sequence
  Artificial, non-naturally occuring RNA aptamers which bind with selectivity/specificity to biliverdin
uugguucugcuguugggacacuguuguccaugacggcugg

SEQ ID NO: 183 - RNA, 40 nt, Artificial Sequence
  Artificial, non-naturally occuring RNA aptamers which bind with selectivity/specificity to biliverdin
uugucagcucgcgucgauagucuaguacgguguauggggu

SEQ ID NO: 184 - RNA, 40 nt, Artificial Sequence
  Artificial, non-naturally occuring RNA aptamers which bind with selectivity/specificity to biliverdin
uuugauugacggauucugaugccgaacacacgucgcgugg

SEQ ID NO: 185 - RNA, 40 nt, Artificial Sequence
  Artificial, non-naturally occuring RNA aptamers which bind with selectivity/specificity to biliverdin
uuugcaagcuuaugaguagcacuucggaauccuugcagcu

(19) United States
(12) Patent Application Publication
(10) Pub. No.: US 2018/0356408 A1
(43) Pub. Date: Dec. 13, 2018

(54) METHODS AND MATERIALS FOR SENSITIVE DETECTION OF TARGET MOLECULES

(71) Applicants: Base Pair Biotechnologies, Inc., Pearland, TX (US); University of Delaware, Newark, DE (US)

(72) Inventors: George W. Jackson, Pearland, TX (US); Jeffrey Caplan, Newark, DE (US); Kun Huang, Newark, DE (US); Blake Meyers, Newark, DE (US)

(21) Appl. No.: 15/777,329

(22) PCT Filed: Nov. 19, 2016
(86) PCT No.: PCT/US16/62965

§ 371 Date: May 18, 2018

Related U.S. Application Data
(60) Provisional application No. 62/257,285, filed on Nov. 19, 2015.
(60) Provisional application No. 62/416,132, filed on Nov. 1, 2016.

(51) Int. Cl. G01N 33/53; C12N 15/115

(52) U.S. Cl. CPC G01N 33/5308; C12N 2320/10; C12N 2310/16; C12N 15/115

(57) ABSTRACT

The subcellular localization of mRNA, and small RNA movement and trafficking of these molecules in an organism are important components of cellular and organismal regulation and communication. The next frontier for analysis of RNA will involve analysis at a single-molecule level, and at the subcellular level. This invention relates to methods and materials for sensitive detection of target molecules, particularly to methods and materials for binding or otherwise associating with target molecules and producing a signal for detection of, for example, spatial and/or temporal localization, and more particularly to methods and materials for the above using aptamers which bind to a chromophore or otherwise produce fluorescence or other detectable signal. This invention further relates to aptamers which bind to chromophores such as, for example, biliverdin or other related molecules, and also to such molecules which exhibit fluorescence or other detectable signals.

### CROSS-REFERENCE TO RELATED APPLICATIONS

This application is a 371 national phase application of Patent Cooperation Treaty (PCT) international application Ser No. PCT/US16/62965, filed Nov. 19, 2016, entitled “Methods and Materials for Sensitive Detection of Target Molecules”, which claims the benefit and priority of the following United States Provisional Patent Applications: Ser. No. 62/257,285, filed Nov. 19, 2015, entitled “A Biliverdin-Binding RNA Aptamer for Fluorescent RNAs and a Small RNA Sensor”, and Ser. No. 62/416,132, filed Nov. 1, 2016, entitled “Functional Ligands to Biliverdin”. The contents of these above listed applications are hereby incorporated by reference in their entireties.

### GOVERNMENT LICENSE RIGHTS

This invention was made with government support under Grant No. 1252939 awarded by the National Science Foundation and Grant Nos. 1R41GM110877 and 1R41GM097811 awarded by the National Institutes of Health. The government has certain rights in the invention.

### FIELD OF THE INVENTION

This invention relates to methods and materials for sensitive detection of target molecules, particularly to methods and materials for binding or otherwise associating with target molecules and producing a signal for detection of, for example, spatial and/or temporal localization, and more particularly to methods and materials for the above using aptamers which bind to a chromophore or otherwise produce fluorescence or other detectable signal. This invention further relates to aptamers which bind to chromophores such as, for example, biliverdin or other related molecules, and also to such molecules which exhibit fluorescence or other detectable signals.

### SEQUENCE LISTING

Ribonucleic acid sequences, which are disclosed in the ASCII text file entitled “P1033PC01_ST25.txt”, created on Nov. 19, 2016 and of 51.5 KB in size, which is incorporated by reference in its entirety, herein are intended to include other aptamers incorporating modifications, truncations, incorporations into larger molecules or complexes, and/or other aptamers having substantial structural or sequence homology, for example, greater than 75% sequence homology, as well as DNA and/or other non-RNA aptamers. The disclosed aptamers may also bind to homologous or related molecules, such as from organisms other than the organisms listed herein, which may include recombinant or non-recombinant versions of the molecules, to modified versions of the molecules, to molecules from sources other than the source listed herein. The indication of the species and source of the target molecules is given for reference only and is not intended to be limiting.

### BACKGROUND OF THE INVENTION

The subcellular localization of small RNAs such as messenger RNA (mRNA) and other small RNAs, and small RNA movement and trafficking of these molecules in an organism are important components of cellular and organismal regulation and communication. RNA is information, and the movement of this information within and between cells, organisms and species is necessary for life. Small RNAs may include, for example and without limitation, microRNA (miRNA), Piwi-interacting RNA (piRNA), small interfering RNA (siRNA), small nucleolar RNA (snoRNAs), tRNA-derived small RNA (tsRNA), small rDNA-derived RNA (srRNA), small nuclear RNA, also commonly referred to as U-RNA, and messenger RNA and portions or RNAs derived from them (mRNA).

However, many studies of RNA, focus on gross calculations of RNA abundance and diversity from large quantities of cells. The next frontier for analysis of RNA will involve analysis at a single-molecule level, and at the subcellular level. In plants, grafting experiments have shown mobile small RNAs are transported from shoot to root, a mechanism of systemic transport of RNA signals for RNA-mediated gene silencing. Specific endogenous RNA molecules, such as those from the leaf sucrose transporter SUT1 and the pumpkin phloem sap mRNA CmNACP, are also transported through the plant vascular system. Enormous siphonous (single-cell, multinucleate) algae such as Caulerpa taxifolia raise questions about RNA trafficking and spatial restriction of transcripts. In mammals, mRNA localization and local translation are seen in polarized, asymmetric cells, such as β-actin mRNA in distal growth cones of neurons cells. Mammalian miRNAs have been found outside of cells, and there is tremendous interest in delivering siRNAs to mammalian tissue in vivo for targeted gene therapy. Cross-species RNA transfer has also been described using genomic tools, for example in recent groundbreaking studies of a parasitic plant (Cuscuta, or dodder) and its host plants, and in the interaction of the fungal pathogen Botrytis cinerea with Arabidopsis and tomato. At a subcellular level, being able to localize and quantify RNA levels, potentially including mRNA transcription and small RNA precursors, together with proteinaceous biogenesis components, will provide breakthroughs in our understanding of RNA.

Apart from gross measurements conferred by so-called next-generation sequencing or microarrays, current methods for the detection, localization and/or quantification of RNA include northern blots, silencing GFP sensor, and in situ hybridization. Northern blotting is an in vitro method that separates RNA molecules according to size using denaturing PAGE (dPAGE), followed by transfer and detection on a nylon membrane. It usually requires a large input of total RNA relative to the molecule of interest. Subcellular- or tissue-focused localization of certain abundant RNAs can be studied by extracting RNAs from different organelles or cell types purified by lasercapture microdissection (LCM). However, LCM fails to capture sufficient RNA for most sequencing-based measurements of small RNA. To provide more precise measurements, in situ hybridization can localize small RNAs in plant and animal tissues, often using locked nucleic acid (LNA) oligonucleotide probes. This approach has successfully shown the localization of small RNAs in the maize shoot apex, mouse brain, and Drosophila embryos, and, by us, in the maize anther. However, in situ hybridization requires tissue fixation, embedding and sectioning, as well as extensive process steps; due to the fixation, it cannot be used to study the dynamics of small RNA processing and movement or the three dimensional (3D) distribution in organs or whole organisms. Yet another strategy for small RNA analysis and localization is to develop sensors that measure the silencing of GFP.

The Jaffrey lab at the Weill Medical College of Cornell University developed a short RNA sequence known as “Spinach” (the reference to a plant is apparently entirely coincidental, as they have not demonstrated utility in a plant). This method made possible the direct fluorescent tagging of mRNAs, via fusion of a target RNA with the 84-nt Spinach sequence. The RNA fluorophore complex exhibited strong and consistent fluorescence under an excitation wavelength upon binding of the fluorophore DFHBI (3,5-difluoro-4-hydroxybenzylidene imidazolinone). The co-crystal structure of Spinach bound to DFHBI showed Spinach-induced fluorescence by forming G-quadruplexes. However, signal from Spinach in live animal or plant cells has been difficult to produce or detect.

Aptamers, which are non-naturally occurring nucleic acids and nucleic acid analogs, are used as ligands capable of binding to molecular targets, and have recently attracted increased attention for their potential application in many areas of biology and biotechnology. In general, aptamers are selected for their specific binding affinity to a particular target molecule, which may, without being bound to any particular theory, be an interaction with a small dissociation constant (K_{d}) preferably on the order of less than 500 nM, with specificity and/or selectivity for the target molecule in the presence of other molecules or subsets of molecules (i.e. greatly preferential binding to the target molecule over others in general or others within a particular system or situation). Aptamers may also be useful by interacting with a target molecule in a manner not present in naturally occurring systems or situations, such as by, for example, not being already present or having a pre-existing function in a naturally occurring setting. They may be used as sensors, therapeutic tools, to regulate cellular processes, as well as to guide drugs to their specific cellular target(s). Contrary to the actual genetic material, their specificity and characteristics are not directly determined by their primary sequence, but instead by their secondary and/or tertiary structure. Aptamers have been recently investigated as immobilized capture elements in a microarray format. Others have recently selected aptamers against whole cells and complex biological mixtures. Aptamers may also, for example, exhibit changes in their secondary and/or tertiary structure depending on whether it is complexed or uncomplexed with a target molecule.

Aptamers are commonly identified by an in vitro method of selection sometimes referred to as Systematic Evolution of Ligands by EXponential enrichment or “SELEX”. SELEX typically begins with a very large pool of randomized polynucleotides which is generally narrowed to one aptamer ligand per molecular target. Once multiple rounds (typically 10-15) of SELEX are completed, the nucleic acid sequences are identified by conventional cloning and sequencing. Aptamers have most famously been developed as ligands to important proteins, rivaling antibodies in both affinity and specificity, and the first aptamer-based therapeutics are now emerging. More recently, however, aptamers have been also developed to bind small organic molecules and cellular toxins, viruses, and even targets as small as heavy metal ions.

### SUMMARY OF THE INVENTION

This invention relates to methods and materials for sensitive detection of target molecules, particularly to methods and materials for binding or otherwise associating with target molecules and producing a signal for detection of, for example, spatial and/or temporal localization, and more particularly to methods and materials for the above using aptamers which bind to a chromophore or otherwise produce fluorescence or other detectable signal. This invention further relates to aptamers which bind to chromophores such as, for example, endogenously present chromophores in target organisms, such as biliverdin, Flavin mononucleotide or other related molecules, and also to such molecules which exhibit fluorescence or other detectable signals. Biliverdin, as used herein, may generally be understood to include, without limitation, similar molecules, derivative molecules and/or versions of biliverdin as they may occur in different organisms. Biliverdin may also be referred to as, for example and without limitation: 3,18-Diethenyl-1,19,22,24-tetrahydro-2,7,13,17-tetrametyl-1,19-dioxo-21H-biline-8,12-dipropanoic Acid; Biliverdine; Billiverdine; Billiverdin; Biliverdin IXa; Dehydrobilirubin; NSC 62793; Oocyan; Protobiliverdin IXa; Uteroverdine; and α-Biliverdin.

In one aspect of the present invention, a method for sensitive detection of target molecules includes a molecular sensor which includes a portion that interacts with a chromophore to produce a measurable and/or detectable signal, such as fluorescence. For example, a molecular sensor may include a portion that interacts with a chromophore, such as by intermolecular binding or similar mechanism(s), and generates fluorescence through the interaction.

In some exemplary embodiments, a molecular sensor may include or be formed/derived from a nucleic acid or nucleic acid analog, such as an aptamer, which exhibits specific binding activity for a chromophore and may further exhibit a luminescence change, such as fluorescence, phosphorescence or other signal emission, such as a chromatic or other change in emission or absorption, due to the interaction with the chromophore. Aptamers may be particularly desirable due to their non-naturally occurring sequence, which may reduce the probability of existing function in natural systems, and their specificity for their target molecules. Nucleic acids may also be desirable as they may be expressed in vivo and thus may be utilized without introducing exogenous material after an initial stage.

For example, a molecular sensor for detecting a target molecule comprises a nucleic acid comprising a first chromophore binding region (CBR) comprising an aptamer sequence with specific binding affinity for a chromophore, said chromophore being biliverdin.

For further example, a molecular sensor for detecting a target molecule comprises a nucleic acid comprising a first CBR comprising an aptamer sequence with specific binding affinity for a chromophore; and a target molecule binding region (TBR) comprising a nucleic acid sequence selected to interact with a target small RNA by hybridization; wherein a detectable signal is generated upon binding of said chromophore to said CBR.

For another example, a molecular sensor for detecting a target molecule comprises a nucleic acid comprising a first CBR comprising an aptamer sequence with specific binding affinity for a chromophore, said chromophore being biliverdin; wherein a detectable signal is generated upon binding of said chromophore to said CBR.

In some exemplary embodiments, the aptamer or other nucleic acid may be selected for specific binding to a chromophore which is endogenous or otherwise typically present in usable quantities in a system of interest, such as in a cell or tissue. Such chromophores may be desirable as they do not require exogenous introduction to be utilized with the molecular sensor and since some chromophores may not be easily or practically introduced into certain systems, such as into cells or tissues where they are at least partially impermeable to exogenous chromophores. In some embodiments, the aptamer or other nucleic acid may be selected for specific binding to biliverdin, which is present in cells in a diverse set of organisms, including mammals, plants and fungi.

For example, a molecular sensor for detecting a target molecule comprises a nucleic acid comprising a first CBR comprising an aptamer sequence with specific binding affinity for a chromophore, said chromophore being endogenous in a cell; and a TBR comprising a nucleic acid sequence selected to interact with a target small RNA by hybridization; wherein a detectable signal is generated upon binding of said chromophore to said CBR.

In some exemplary embodiments, a molecular sensor may act as a switch in the presence of both a target molecule and a chromophore. In some embodiments, a molecular sensor may be nucleic acid which includes a region which may be based on or derived from an aptamer which may bind to a chromophore, and further includes a region which may bind, hybridize or otherwise interact with a target molecule of interest in a manner that causes a conformational or other structural shift or change in the nucleic acid. For example, the nucleic acid may exist in a state where the chromophore binding region is in a state where it does not bind the chromophore and the subsequent interaction between the nucleic acid with the target molecule may enable a binding event between the chromophore and the nucleic acid to, for example, produce a signal. For example, the binding of the chromophore may result in the nucleic acid or a portion thereof to fluoresce or otherwise enable emission of a signal. This may also be desirable to colocalize the chromophore and the target molecule through both binding to the molecular sensor.

For example, a molecular sensor for detecting a target molecule comprises a nucleic acid comprising: first CBR comprising an aptamer sequence with specific binding affinity for a chromophore; and a TBR comprising a nucleic acid sequence selected to interact with a target small RNA by hybridization; wherein a detectable signal is generated upon binding of said chromophore to said CBR and upon interaction between said TBR and said target small RNA.

For further example, a molecular sensor for detecting a target molecule comprises a nucleic acid comprising: a first CBR comprising an aptamer sequence with specific binding affinity for a chromophore, said chromophore being endogenous in a cell; and a TBR comprising a nucleic acid sequence selected to interact with a target small RNA in said cell by hybridization; wherein a detectable signal is generated upon binding of said chromophore to said CBR and upon interaction between said TBR and said target small RNA.

For another example, a molecular sensor for detecting a target molecule comprises a nucleic acid comprising a first CBR comprising an aptamer sequence with specific binding affinity for a chromophore, said chromophore being biliverdin; and a TBR comprising a nucleic acid sequence selected to interact with a target molecule; wherein a detectable signal is generated upon binding of said chromophore to said CBR and upon interaction between said TBR and said target molecule.

In some embodiments, a linker(s) or other additional sequence regions may be included to enhance or enable the response to the target molecule interaction. For example, a region which forms a stem-hybridized structure upon the interaction with the target molecule may be utilized to induce a desired conformational change in the chromophore-binding region to enable binding.

In some embodiments, the molecular sensor may further include a ratiometric or other feature to, for example, aid in quantification of the molecular sensors and/or for ratiometric measurement of the quantity of molecular sensors and the quantity of target molecules associated with the molecular sensors. For example, additional chromophore binding regions which constitutively emit signal independent of the target molecule interaction may be utilized.

The molecular sensor may generally further include a region which interacts with a target molecule, such as RNAs, DNAs, proteins, cellular structures, metabolites, and/or any other desired molecular target. In some exemplary embodiments, a molecular sensor may include a region with a nucleic acid sequence which hybridizes to complementary nucleic acids which may be present, such as in a cell or tissue. This may be desirable to study spatial and temporal localization of such nucleic acids in a cell or tissue by their interaction with the molecular sensor. This may also be desirable to colocalize the chromophore and the target molecule through both binding to the molecular sensor.

In some embodiments, a template for a molecular sensor may be encoded into a nucleic acid and introduced into a cell such that the cell expresses the template to generate the molecular sensor in situ. For example, a template may be encoded into a plasmid or other expression package which may generally include a promoter and/or other elements to enable its expression in a target cell or cell type. The expression package may then be introduced into the cell of interest, such as by transfection, transduction, transformation and/or any other appropriate method. This may be desirable as the molecular sensor may be generated in the cell of interest and may be protected from extracellular factors which may damage or otherwise negatively affect the molecular sensor.

For example, a method for detecting target molecules, such as small RNAs, proteins or other cellular components, in a cell comprises introducing an expression package into a cell, said expression package comprising a double-stranded DNA incorporating an operative promoter coupled to a gene encoding a nucleic acid transcript comprising a first CBR comprising an aptamer sequence with specific binding affinity for an endogenous chromophore and a TBR comprising a nucleic acid sequence selected to interact with a target molecule, such as by hybridization or through non-Watson Crick interactions, wherein a fluorescent signal is generated upon binding of said chromophore to said CBR and upon interaction between said TBR and said target molecule; expressing said expression package in said cell; and detecting said fluorescent signal to determine the presence of said target molecule in said cell.

In some embodiments, the nucleic acid of the molecular sensor may also be modified and/or include portions which aid in protecting the molecular sensor from degradation and/or interaction with undesirable elements present in a system, such as, for example, RNA-binding or other nucleic acid-binding proteins which may, for example, quench fluorescence and/or otherwise interfere with generation of detectible signals. For example, the aptamers may be linked to a guide RNA or similar molecule that binds to a chosen RNA-binding protein, such as, for example, dCas9, MS2 or other proteins, as a protecting feature for the molecular sensor to prevent other, unwanted RNA-binding proteins or elements from binding or otherwise influencing the molecular sensor. In other examples, the aptamers may hybridize to or include a portion that hybridizes to a nucleic acid tag which may be attached to a protective feature, such as, for example, a bead, an RNA-binding protein, a modified nucleotide and/or any other appropriate feature which may aid in protecting the aptamer from unwanted RNA-binding proteins.

In some embodiments, the molecular sensor may also include features for controlling localization of the expression package within the genome of a host cell and/or the resulting molecular sensor generated from the template to particular portions of the cell, such as within the nucleus or in the cytosol. For example, localization signals may be incorporated into dCas9 or into an sgRNA.

In a further aspect, molecular sensors as discussed above may be utilized for live cell or fixed sample imaging or visualization.

In another aspect, a molecular sensor may be directly attached to a target molecule of interest. In some embodiments, an aptamer or other nucleic acid ligand may be expressed as an appended sequence to a nucleic acid sequence of interest in a cell or tissue, such as to an mRNA transcript or other small RNA expressed in a cell. The appended sequence may then be utilized to detect the sequence of interest by emitting a signal, such as by binding to a chromophore such as biliverdin and fluorescing.

In another aspect of the present invention, functional ligands may be generated, selected and/or utilized which bind with specificity and to chromophores, and more particularly, for example, to chromophores which are endogenous in living cells or tissues, such as, for example, biliverdin. In some exemplary embodiments, nucleic acid or nucleic acid analog ligands may be utilized, such as aptamers. Aptamers are generally selected from large pools or libraries of randomized short nucleic acids, such as ribonucleic acid (RNA), deoxyribonucleic acid (DNA), artificially modified nucleic acids, and/or combinations thereof, through a repetitive process referred to as SELEX and its variants.

In general, aptamers may be selected by exposing pools or libraries of nucleic acids to a target molecule during SELEX to screen for binding activity, such as with chromophores including biliverdin. Aptamers may further have advantages over other forms of specific ligands, such as antibodies, due to their small size, ability to be relatively easily synthesized, ability to be relatively easily expressed in vivo and ability to recognize small molecular targets.

In some exemplary embodiments, aptamers may be selected and screened for signal emitting qualities, such as, for example, upon binding to their target molecules. For example, nucleic acids or analogs may be screened for binding activity via a SELEX protocol to a target molecule and further screening may be conducted by expressing candidate aptamers in a cell. In some embodiments where the target molecule is endogenous to the host cell, signal emission may be directly measured after inserting an aptamer expression package into the host cell. For example, biliverdin may be endogenously present in a variety of host cells, such as yeast and other eukaryotic cells which may be desirable for conducting rapid insertion of aptamer expression package libraries for subsequent signal detection, such as by detecting and/or measuring potential fluorescence due to aptamer binding to biliverdin.

For example, a method for selecting molecular sensor sequences comprises introducing an expression package into a cell containing an endogenous chromophore, said expression package comprising a double-stranded DNA incorporating an operative promoter coupled to a gene encoding a nucleic acid transcript comprising a first CBR comprising an aptamer sequence with specific binding affinity for said endogenous chromophore; expressing said expression package in said cell; interrogating said cell for a fluorescence signal generated upon said first CBR binding to said endogenous chromophore; and sorting said cell if said fluorescence signal is detected.

The present invention together with the above and other advantages may best be understood from the following detailed description of the embodiments of the invention and as illustrated in the drawings. The following description, while indicating various embodiments of the invention and numerous specific details thereof, is given by way of illustration and not of limitation. Many substitutions, modifications, additions or rearrangements may be made within the scope of the invention, and the invention includes all such substitutions, modifications, additions or rearrangements.

### BRIEF DESCRIPTION OF THE FIGURES

FIG. 1 illustrates an embodiment of a molecular sensor with a chromophore binding region binding a chromophore and emitting a signal;

FIG. 2 illustrates an embodiment of a molecular sensor with a chromophore binding region and a target binding region, where the binding of a target molecule induces a conformation change of the chromophore binding region to enable binding of a chromophore and signal;

FIG. 3 illustrates an embodiment of a molecular sensor with multiple chromophore binding regions which emit different signals;

FIG. 4 illustrates a protective feature on a molecular sensor;

FIG. 5 shows sorting of cells expressing molecular sensors based on signal emission;

FIG. 6 illustrates MST binding data for a chromophore binding aptamer; and

FIGS. 7 and 7a illustrate chromophore binding aptamer candidates expressed in E. coli and measured/observed fluorescence.

### DETAILED DESCRIPTION OF THE INVENTION

The detailed description set forth below is intended as a description of the presently exemplified methods, devices, and compositions provided in accordance with aspects of the present invention and is not intended to represent the only forms in which the present invention may be practiced or utilized. It is to be understood, however, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the exemplified methods, devices and materials are now described.

This invention relates to methods and materials for sensitive detection of target molecules, particularly to methods and materials for binding or otherwise associating with target molecules and producing a signal for detection of, for example, spatial and/or temporal localization, and more particularly to methods and materials for the above using aptamers which bind to a chromophore or otherwise produce fluorescence or other detectable signal. This invention further relates to aptamers which bind to chromophores such as, for example, biliverdin or other related molecules, and also to such molecules which exhibit fluorescence or other detectable signals.

In one aspect of the present invention, a method for sensitive detection of target molecules includes a molecular sensor which includes a portion that interacts with a chromophore to produce a measurable and/or detectable signal, such as fluorescence. For example, a molecular sensor may include a portion that interacts with a chromophore, such as by intermolecular binding or similar mechanism(s), and generates fluorescence through the interaction.

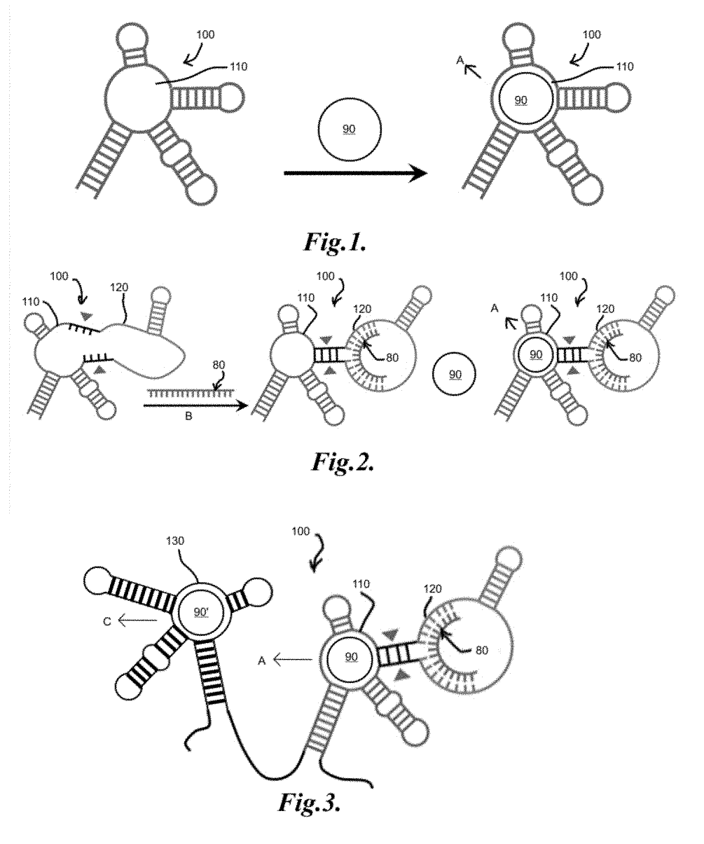

FIG. 1 illustrates an example of a molecular sensor 100 which includes a chromophore binding region 110 that emits a signal A, such as fluorescence, after binding to a chromophore 90.

In some exemplary embodiments, a molecular sensor may include or be formed/derived from a nucleic acid or nucleic acid analog, such as an aptamer, which exhibits specific binding activity for a chromophore and further exhibits an optical property change, either in itself or of the chromophore, the molecular sensor as a whole or some subcombination thereof. These may include a luminescence change, such as fluorescence, phosphorescence or other signal emission, such as a chromatic or other change in emission or absorption, due to the interaction with the chromophore. Aptamers may be particularly desirable due to their non-naturally occurring sequence, which may reduce the probability of existing function in natural systems, and their specificity for their target molecules. Nucleic acids may also be desirable as they may be expressed in vivo and thus may be utilized without introducing exogenous material after an initial stage. In general, aptamers achieve their binding activity through their secondary and/or tertiary structure in the appropriate environment and conditions. Without being bound to any particular theory, the nucleic acid of the aptamer can form one or more stems (i.e., base-paired regions) as well as one or more non base-paired regions along the length of the stem. These non base-paired regions can be in the form of a bulge or loop (e.g., internal loop) along the length of the stem(s) and/or a loop at the end of the one or more stem(s) (e.g., hairpin loop). These nucleic acid aptamers possess specificity in binding to a particular target molecule, and they non-covalently bind their target molecule through an interaction such as an ion-ion force, dipole-dipole force, hydrogen bond, van der Waals force, electrostatic interaction, stacking interaction and/or any combination of these interactions in a non-Watson-Crick binding interaction between the aptamer and its target molecule.

The molecular sensor including an aptamer or other nucleic acid sequence may, for example, be in the form of a single-stranded nucleic acid, a fully or partly double-stranded nucleic acid, a nucleic acid complex with a protein or other molecule(s), nucleic acids covalently bound to other molecule(s), multiple separate nucleic acids having differing degrees of hybridization to each other, and/or any other appropriate nucleic acid formation or structure or combinations thereof. Nucleic acids may include naturally-occurring biomolecules such as ribonucleic acid (RNA) and deoxyribonucleic acid (DNA), artificially modified nucleic acids, and/or combinations thereof.

In examples of fluorescence, the aptamer, the chromophore and/or the molecular sensor as a whole may generally exhibit low or even no fluorescence in the absence of binding between the aptamer and the chromophore. In general, it may be desirable for the quantum yield without binding between the aptamer and the chromophore to be as low as possible, such as, for example, less than about 0.01, more particularly less than about 0.001, further particularly less than about 0.0001. It may further be desirable to choose a chromophore which does not autofluoresce significantly or produce fluorescence from interactions with other molecules besides the chosen aptamer in situ.

In some exemplary embodiments, the aptamer or other nucleic acid may be selected for specific binding to a chromophore which is endogenous or otherwise typically present in usable quantities in a system of interest, such as in a cell or tissue. Such chromophores may be desirable as they do not require exogenous introduction to be utilized with the molecular sensor and since some chromophores may not be easily or practically introduced into certain systems, such as into cells or tissues where they are at least partially impermeable to exogenous chromophores. In some embodiments, the aptamer or other nucleic acid may be selected for specific binding to biliverdin.

Biliverdin is a tetrapyrrolic pigment which is present in numerous eukaryotic and some prokaryotic organisms, such as in microbes, fungi, plants, avian egg shells, the blue-green blood of many marine fish, the blood of tobacco hornworm, the wings of moth and butterfly, the serum and eggs of frogs, and the placenta of dogs. In the garfish (Belone belone) and related species, the bones are bright green because of biliverdin. In humans, it is generally the result from the breakdown of the heme moiety of hemoglobin in erythrocytes. Macrophages break down senescent erythrocytes and break the heme down into biliverdin, which normally rapidly reduces to free bilirubin. Biliverdin is seen briefly in some bruises as a green color. In bruises, its breakdown into bilirubin leads to a yellowish color. It further acts as a chromophore and can participate in fluorescence. Biliverdin may also be referred to as, for example and without limitation: 3,18-Diethenyl-1,19,22,24-tetrahydro-2,7,13,17-tetrametyl-1,19-dioxo-21H-biline-8,12-dipropanoic Acid; Biliverdine; Billiverdine; Billiverdin; Biliverdin IXa; Dehydrobilirubin; NSC 62793; Oocyan; Protobiliverdin IXa; Uteroverdine; and a-Biliverdin.

In some embodiments, aptamers to biliverdin may be utilized which, upon binding between the aptamer and biliverdin, fluorescence may result, such as red, far-red or near-IR fluorescence. Fluorescence in this range may be desirable, for example, in imaging in whole mount, cleared plant samples. For example, once cleared, there is typically less autofluorescence in that spectral range and far-red may generally be superior for deep tissue imaging in plants and animals.

In some exemplary embodiments, a molecular sensor may act as a switch in the presence of both a target molecule and a chromophore. In some embodiments, a molecular sensor may be nucleic acid which includes a region which may be based on or derived from an aptamer which may bind to a chromophore, and further includes a region which may bind, hybridize or otherwise interact with a target molecule of interest in a manner that causes a conformational or other structural shift or change in the nucleic acid.

FIG. 2 illustrates an example of a molecular sensor 100 which changes conformation upon interaction with a target molecule 80, illustrated with a complementary small RNA, with a target binding region 120, which may result in a conformational change B in the chromophore binding region 110, enabling it to bind to chromophore 90 and emit a signal A, such as fluorescence. For example, the nucleic acid may exist in a state where the chromophore binding region, which may be an or be derived from an aptamer to the chromophore, is in a state where it does not bind the chromophore and the subsequent interaction between the nucleic acid with the target molecule may enable a binding event between the chromophore and the nucleic acid to, for example, produce a signal. For example, the binding of the chromophore may result in the nucleic acid or a portion thereof to fluoresce or otherwise enable emission of a signal. In some embodiments, a linker(s) or other additional sequence regions may be included to enhance or enable the response to the target molecule interaction. For example, a region which forms a stem-hybridized structure upon the interaction with the target molecule may be utilized to induce a desired conformational change in the chromophore-binding region to enable binding. FIG. 2 illustrates the use of a feature 102, such as the transducer stem forming region illustrated (triangle arrows), which responds to the binding of the target molecule 80 to form and/or stabilize the binding conformation of the chromophore binding region 110, which may be aptamer derived from an aptamer. This may also be desirable to colocalize the chromophore and the target molecule through both binding to the molecular sensor. In this manner, for example, the molecular sensor may act as a colocalizing agent between the chromophore and the target molecule such that, for example, the target molecule and chromophore may interact, such as to produce a signal. For further example, the molecular sensor itself may not generate any particular signal, but the signal may originate from the interaction between the chromophore and the target molecule through proximity or other mechanism mediated by the molecular sensor.

In some embodiments, the molecular sensor may further include a ratiometric or other feature to, for example, aid in quantification of the molecular sensors and/or for ratiometric measurement of the quantity of molecular sensors and the quantity of target molecules associated with the molecular sensors. For example, as illustrated in FIG. 3, a ratiometric molecular sensor 100′ may include a first chromophore binding region 110, a target binding region 120 and a second chromophore binding region 130. In general, one of the chromophore binding regions may bind its chromophore independently of the target molecule binding to the sensor, and thus may constitutively emit a signal in situ to provide, for example, quantitation of the molecular sensor number, whereas the other chromophore binding region may generally only emit signal after the target binding region 120 binds to a target molecule to induce the proper conformational change to allow it to bind its chromophore. This may also aid in temporal localization of target molecules by monitoring the position and time that the specific chromophore binding regions being emitting a signal. In some embodiments, additional chromophore binding regions and/or additional target binding regions may also be included to produce differing signal emission effects based on binding events for each region or together. In general, the different chromophore binding regions may be selected or designed to emit different signals from each other to discriminate, such as different wavelength fluorescence binding the same chromophore. They may also bind different chromophores, such as, for example, other endogenous chromophores including light-oxygen-voltage sensing domain (LOV-domain) fluorescent proteins, such as flavin mononucleotide (FMN), and may be selected for different emissions.

The molecular sensor may generally further include a region which interacts with a target molecule, such as RNAs, DNAs, proteins, cellular structures, metabolites, and/or any other desired molecular target. In some exemplary embodiments, a molecular sensor may include a region with a nucleic acid sequence which hybridizes to complementary nucleic acids which may be present, such as in a cell or tissue. This may be desirable to study spatial and temporal localization of such nucleic acids, such as small RNAs, in a cell or tissue by their interaction with the molecular sensor. In other embodiments, the region may include an aptamer or aptamer derived sequence which may be utilized to bind non-nucleic acid target molecules. As discussed above, the sequence may be utilized to create a switching effect in the molecular sensor, such as by its location, size or other design factor within the nucleic acid. This may also be desirable to colocalize the chromophore and the target molecule through both binding to the molecular sensor.

In some embodiments, a template for a molecular sensor may be encoded into a nucleic acid and introduced into a cell such that the cell expresses the template to generate the molecular sensor in situ. For example, a template may be encoded into a plasmid or other expression package which may generally include a promoter and/or other elements to enable its expression in a target cell or cell type. The expression package may then be introduced into the cell of interest, such as by transfection, transduction, transformation and/or any other appropriate method. This may be desirable as the molecular sensor may be generated in the cell of interest and may be protected from extracellular factors which may damage or otherwise negatively affect the molecular sensor. In general, the selection of expression package may depend on the cell or cell type of interest and a wide variety of expression packages are commercially available. It may further be generally understood in the art how to utilize standard molecular biology methods and techniques to incorporate the desired elements of a molecular sensor into an expression package and to introduce them into a cell of interest.

In some embodiments, the nucleic acid of the molecular sensor may also be modified and/or include portions which aid in protecting the molecular sensor from degradation and/or interaction with undesirable elements present in a system, such as, for example, RNA-binding or other nucleic acid-binding proteins which may, for example, quench fluorescence and/or otherwise interfere with generation of detectible signals. For example, the aptamers or other portions of the molecular sensor may be linked to a guide RNA or similar molecule that binds to a chosen RNA-binding protein, such as, for example, dCas9, MS2 or other proteins, as a protecting feature for the molecular sensor to prevent other, unwanted RNA-binding proteins or elements from binding or otherwise influencing the molecular sensor. In other examples, the aptamers may hybridize to or include a portion that hybridizes to a nucleic acid tag which may be attached to a protective feature, such as, for example, a bead, an RNA-binding protein, a modified nucleotide and/or any other appropriate feature which may aid in protecting the aptamer from unwanted RNA-binding proteins.

FIG. 4 illustrates an example of a molecular sensor 100″ where the chromophore binding region 110 is incorporated into the context of an RNA with a portion that binds to an RNA-binding protein, as illustrated with sgRNA 140 which may bind to dCas9 (shown as region 70) to provide, for example, protection from undesired RNA-binding proteins which may interfere with the chromophore binding region 110, such as by quenching fluorescence.

In some embodiments, the molecular sensor may also include features for controlling localization of the expression package within the genome of a host cell and/or the resulting molecular sensor generated from the template to particular portions of the cell, such as within the nucleus or in the cytosol. For example, localization signals may be incorporated into dCas9 or into an sgRNA.

In a further aspect, molecular sensors as discussed above may be utilized for live cell or fixed sample imaging or visualization. In general, a suitable radiation source is used to illuminate the molecular sensor in situ to provide input energy for fluorescence or other signal emission, such as from the aptamer:chromophore complex. The radiation source may be used alone or with optical fibers and/or any other appropriate optical waveguide to illuminate the sample. Suitable radiation sources may include, without limitation, filtered, wide-spectrum light sources (e.g., tungsten, or xenon arc), laser light sources, such as gas lasers, solid state crystal lasers, semiconductor diode lasers (including multiple quantum well, distributed feedback, and vertical cavity surface emitting lasers), dye lasers, metallic vapor lasers, free electron lasers, and/or lasers using any other substance as a gain medium. Common gas lasers may include Argon-ion, Krypton-ion, and mixed gas (e.g., Ar Kr) ion lasers, emitting at 455, 458, 466, 476, 488, 496, 502, 514, and 528 nm (Ar ion); and 406, 413, 415, 468, 476, 482, 520, 531, 568, 647, and 676 nm (Kr ion). Also included in gas lasers are Helium Neon lasers emitting at 543, 594, 612, and 633 m. Typical output lines from solid state crystal lasers include 561 nm ([532 nm (doubled Nd:YAG) and 408/816 nm (doubled/primary from Ti: Sapphire). Typical output lines from semiconductor diode lasers are 635, 650, 670, and 780 mm. Infrared radiation sources may also be employed.

Excitation wavelengths and emission detection wavelengths may vary depending on both the chromophore and the nucleic acid aptamer molecule that are being employed. Some examples of different aptamer:fluorophore combinations are described in PCT Application Publ. No. WO/2010/096584, which is hereby incorporated by reference in its entirety.

Detection of the emission spectra may be achieved using any suitable detection system. In some exemplary embodiments, detection systems may include, without limitation, a cooled CCD camera, a cooled intensified CCD camera, a single-photon-counting detector (e.g., PMT or APD), dual-photon counting detector, spectrometer, fluorescence activated cell sorting (FACS) systems, fluorescence plate readers, fluorescence resonance energy transfer, and/or any other methods that detect photons released upon signal emission, such as fluorescence or other resonance energy transfer excitation of molecules.

In some embodiments, cells or tissues may be permeabilized to introduce molecular sensors and/or chromophores into their interior to bind to target molecules of interest, such as small RNAs. Molecular sensors may also be introduced by electrophoresis, nanoparticle delivery and/or any other appropriate method for introducing exogenous material into cells or tissues. However, without being bound to any particular theory, permeabilization may also result in leakage of target molecules from the cells/tissues of interest.

In some exemplary embodiments, molecular sensors are expressed in the cells of interest, as above, and thus permeabilization may not be necessary for introduction of the molecular sensor.

In another aspect, a molecular sensor may be directly attached to a target molecule of interest. In some embodiments, an aptamer or other nucleic acid ligand may be expressed as an appended sequence to a nucleic acid sequence of interest in a cell or tissue, such as to an mRNA transcript or other small RNA expressed in a cell. The appended sequence may then be utilized to detect the sequence of interest by emitting a signal, such as by binding to a chromophore such as biliverdin and fluorescing. For example, CRISPR-Cas9 genome modification may be utilized to produce targeted “knock-in” introduction of molecular sensor templates such that they are appended to the target transcript. The use of aptamer and other nucleic acid-based molecular sensors may be further desirable in such applications as no “in-frame” considerations are required and aptamer sequences are typically very short (e.g. >50 nt) and may thus have a likelihood of not significantly affecting the transcript of interest.

In another aspect of the present invention, functional ligands may be generated, selected and/or utilized which bind with specificity and to chromophores, and more particularly, for example, to chromophores which are endogenous in living cells or tissues, such as, for example, biliverdin. In some exemplary embodiments, nucleic acid or nucleic acid analog ligands may be utilized, such as aptamers. Aptamers are generally selected from large pools or libraries of randomized short nucleic acids, such as ribonucleic acid (RNA), deoxyribonucleic acid (DNA), artificially modified nucleic acids, and/or combinations thereof, through a repetitive process referred to as SELEX and its variants.

In general, functional ligands such as aptamers may generally include nucleic acids, particularly single-stranded nucleic acids, peptides, other biopolymers and/or combinations or modifications thereof. Nucleic acid sequences may include naturally-occurring biomolecules such as ribonucleic acid (RNA), deoxyribonucleic acid (DNA), artificially modified nucleic acids, and/or combinations thereof. In general, modified nucleic acid bases may be utilized and may include, but are not limited to, 2′-Deoxy-P-nucleoside-5′-Triphosphate, 2′-Deoxyinosine-5′-Triphosphate, 2′-Deoxypseudouridine-5′-Triphosphate, 2′-Deoxyuridine-5′-Triphosphate, 2′-Deoxyzebularine-5′-Triphosphate, 2-Amino-2′-deoxyadenosine-5′-Triphosphate, 2-Amino-6-chloropurine-2′-deoxyriboside-5′-Triphosphate, 2-Aminopurine-2′-deoxyribose-5′-Triphosphate, 2-Thio-2′-deoxycytidine-5′-Triphosphate, 2-Thiothymidine-5′-Triphosphate, 2′-Deoxy-L-adenosine-5′-Triphosphate, 2′-Deoxy-L-cytidine-5′-Triphosphate, 2′-Deoxy-L-guanosine-5′-Triphosphate, 2′-Deoxy-L-thymidine-5′-Triphosphate, 4-Thiothymidine-5′-Triphosphate, 5-Aminoallyl-2′-deoxycytidine-5′-Triphosphate, 5-Aminoallyl-2′-deoxyuridine-5′-Triphosphate, 5-Bromo-2′-deoxycytidine-5′-Triphosphate, 5-Bromo-2′-deoxyuridine-5′-Triphosphate, 5-Fluoro-2′-deoxyuridine-5′-Triphosphate, 5-Trifluoromethyl-2-deoxyuri dine-5′-Triphosphate, and/or any other appropriate modified nucleic acid base. It may generally be understood that the nucleoside triphosphates (NTPs) listed above may generally refer to any appropriate phosphate of the modified base, such as additionally, for example, monophosphates (NMPs) or diphosphates (NDPs) of the base. Embodiments of the SELEX method may generally be utilized to select or preselect for aptamers to be used in a collection. The basic SELEX protocol and aptamers are described in U.S. Pat. No. 5,270,163, entitled “Methods for identifying nucleic acid ligands,” the entire contents of which are hereby incorporated by reference.

In general, aptamers may be selected by exposing pools or libraries of nucleic acids to a target molecule during SELEX to screen for binding activity, such as with chromophores including biliverdin. Aptamers may further have advantages over other forms of specific ligands, such as antibodies, due to their small size, ability to be relatively easily synthesized, ability to be relatively easily expressed in vivo and ability to recognize small molecular targets.

In some exemplary embodiments, the aptamers may be selected utilizing methods which present the library to a diverse group of different target molecules simultaneously, such as to promote the selection of specific binders versus non-specific binders to a particular target molecule. Methods for simultaneous selection against multiple targets using SELEX are described in U.S. Pat. No. 8,314,052, the contents of which is hereby incorporated by reference in its entirety.

In some embodiments, RNA aptamers may be selected for binding to chromophores such as biliverdin. For example, in situations where the aptamer or a derivative thereof may generally be utilized in an expression package to produce it in a cell or tissue, single-stranded products such as RNA may be desirable for incorporation into a larger single stranded nucleic acid which may hybridize to target molecules such as small RNAs in situ.

In some exemplary embodiments, aptamers may be selected and screened for signal emitting qualities, such as, for example, upon binding to their target molecules. For example, nucleic acids or analogs may be screened for binding activity via a SELEX protocol to a target molecule and further screening may be conducted by expressing candidate aptamers in a cell. In some embodiments where the target molecule is endogenous to the host cell, signal emission may be directly measured after inserting an aptamer expression package into the host cell. For example, biliverdin may be endogenously present in a variety of host cells, such as bacteria, yeast and other eukaryotic cells which may be desirable for conducting rapid insertion of aptamer expression package libraries for subsequent signal detection, such as by detecting and/or measuring potential fluorescence due to aptamer binding to biliverdin.

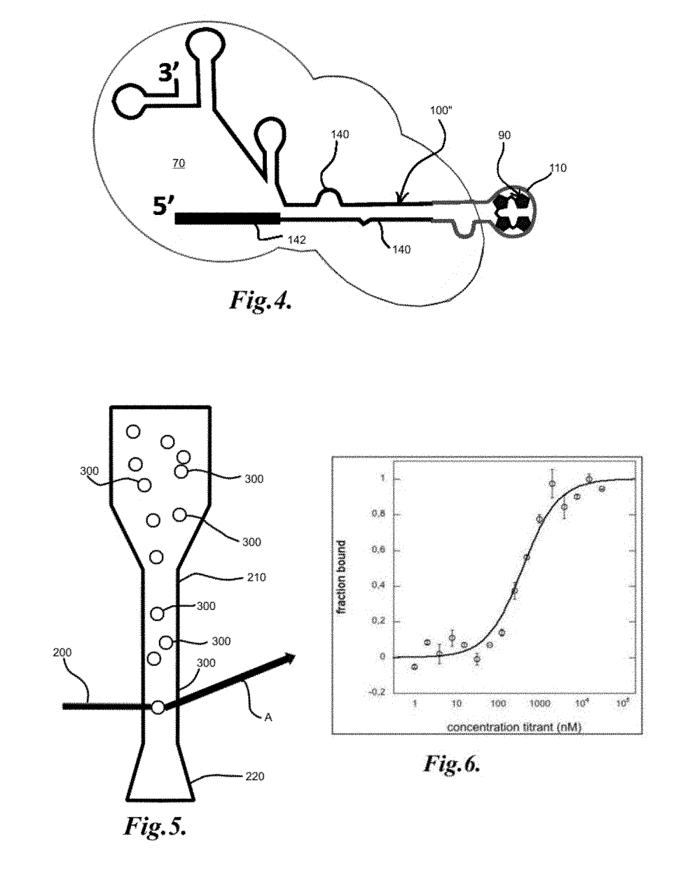

For example, large numbers of cells expressing the candidate library of aptamers may be interrogated with radiation and measured for fluorescence or other signal emission and sorted, such as with high-throughput cell sorting systems, including, but not limited to, fluorescence-activated cell sorting (FACS), as illustrated in FIG. 5. with an excitation source 200 interrogating cells 300 flowing through a channel 210 and sorted based on their fluorescence emissions (e.g. by wavelength, intensity, etc).

### Example of Selex to Select Aptamers for a Chromophore

A variation on a SELEX procedure was performed utilizing biliverdin to produce candidate aptamers, and to yield aptamer sequences given in the sequence listing above. The sequences yielded are artificial, non-naturally occurring sequences designed and/or selected for artificially for specific and/or high affinity binding to utilizing biliverdin and/or similar/related molecules, where the sequences have no known natural function. FIG. 6 illustrates the binding analysis by Microscale Thernophoresis (MST) for a candidate 40 bp RNA aptamer to biliverdin plotted as fraction bound vs. titrated target concentration of biliverdin, showing an average K_{d} of 381 nM. The analysis was performed in 1×PBS with 1 mM MgCl₂ and 0.05% Tween.

### Example of Fluorescence Screening of Biliverdin Aptamers

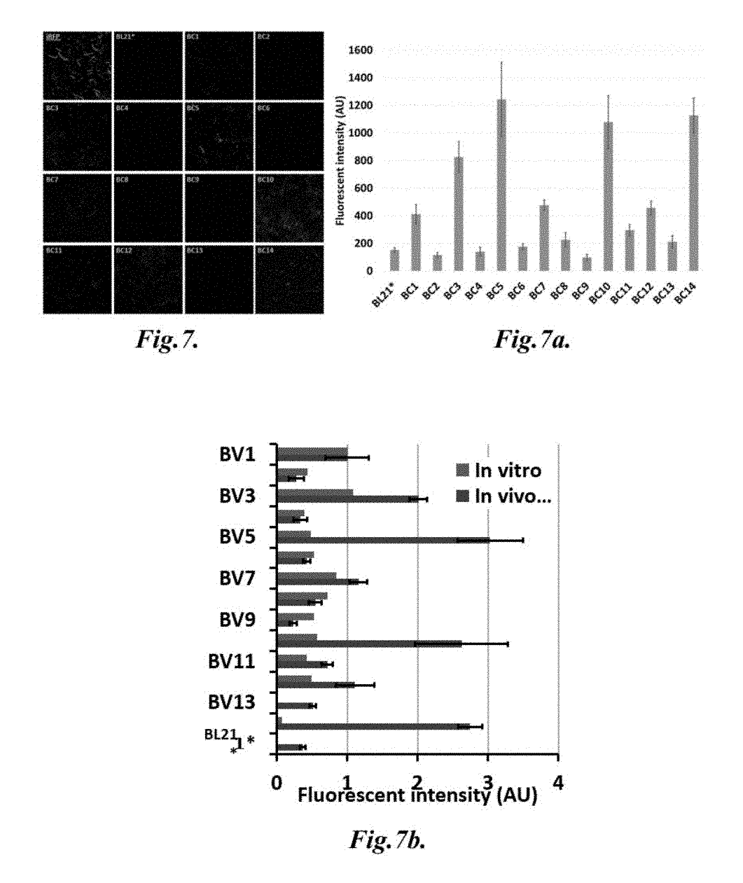

A group of 14 RNA 40-mer aptamers to biliverdin selected by a SELEX procedure were expressed in E. coli and evaluated for fluorescence intensity as illustrated in the fluorescence imaging of candidates BC1-BC14 in FIG. 7 and in the measured fluorescence intensity (AU) graph in FIG. 7a, both using a BL21 E. coli and iRFP control. FIG. 7b illustrates the comparison between the fluorescence intensity (AU) measured from candidates BC1, 3, 5, 7, 9, 11 and 13 vs. the BL21 E. coli control in in vivo and in vitro testing situations.

Although the invention has been described with respect to specific embodiments thereof, these embodiments are merely illustrative, and not restrictive of the invention. The description herein of illustrated embodiments of the invention, including the description in the Abstract and Summary, is not intended to be exhaustive or to limit the invention to the precise forms disclosed herein (and in particular, the inclusion of any particular embodiment, feature or function within the Abstract or Summary is not intended to limit the scope of the invention to such embodiment, feature or function). Rather, the description is intended to describe illustrative embodiments, features and functions in order to provide a person of ordinary skill in the art context to understand the invention without limiting the invention to any particularly described embodiment, feature or function, including any such embodiment feature or function described in the Abstract or Summary. While specific embodiments of, and examples for, the invention are described herein for illustrative purposes only, various equivalent modifications are possible within the spirit and scope of the invention, as those skilled in the relevant art will recognize and appreciate. As indicated, these modifications may be made to the invention in light of the foregoing description of illustrated embodiments of the invention and are to be included within the spirit and scope of the invention. Thus, while the invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the invention.

Reference throughout this specification to “one embodiment”, “an embodiment”, or “a specific embodiment” or similar terminology means that a particular feature, structure, or characteristic described in connection with the embodiment is included in at least one embodiment and may not necessarily be present in all embodiments. Thus, respective appearances of the phrases “in one embodiment”, “in an embodiment”, or “in a specific embodiment” or similar terminology in various places throughout this specification are not necessarily referring to the same embodiment. Furthermore, the particular features, structures, or characteristics of any particular embodiment may be combined in any suitable manner with one or more other embodiments. It is to be understood that other variations and modifications of the embodiments described and illustrated herein are possible in light of the teachings herein and are to be considered as part of the spirit and scope of the invention.

In the description herein, numerous specific details are provided, such as examples of components and/or methods, to provide a thorough understanding of embodiments of the invention. One skilled in the relevant art will recognize, however, that an embodiment may be able to be practiced without one or more of the specific details, or with other apparatus, systems, assemblies, methods, components, materials, parts, and/or the like. In other instances, well-known structures, components, systems, materials, or operations are not specifically shown or described in detail to avoid obscuring aspects of embodiments of the invention. While the invention may be illustrated by using a particular embodiment, this is not and does not limit the invention to any particular embodiment and a person of ordinary skill in the art will recognize that additional embodiments are readily understandable and are a part of this invention.

As used herein, the terms “comprises,” “comprising,” “includes,” “including,” “has,” “having,” or any other variation thereof, are intended to cover a non-exclusive inclusion. For example, a process, product, article, or apparatus that comprises a list of elements is not necessarily limited only those elements but may include other elements not expressly listed or inherent to such process, process, article, or apparatus.

Furthermore, the term “or” as used herein is generally intended to mean “and/or” unless otherwise indicated. For example, a condition A or B is satisfied by any one of the following: A is true (or present) and B is false (or not present), A is false (or not present) and B is true (or present), and both A and B are true (or present). As used herein, including the claims that follow, a term preceded by “a” or “an” (and “the” when antecedent basis is “a” or “an”) includes both singular and plural of such term, unless clearly indicated within the claim otherwise (i.e., that the reference “a” or “an” clearly indicates only the singular or only the plural). Also, as used in the description herein, the meaning of “in” includes “in” and “on” unless the context clearly dictates otherwise.

1. A molecular sensor for detecting a target molecule comprising:
a nucleic acid comprising:
a first chromophore binding region (CBR) comprising an aptamer sequence with specific binding affinity for a chromophore; and
a target molecule binding region (TBR) comprising a nucleic acid sequence selected to interact with a target small RNA by hybridization;
wherein a detectable signal is generated upon binding of said chromophore to said CBR.

2. A molecular sensor for detecting a target molecule comprising:
a nucleic acid comprising:
a first chromophore binding region (CBR) comprising an aptamer sequence with specific binding affinity for a chromophore; and
a target molecule binding region (TBR) comprising a nucleic acid sequence selected to interact with a target small RNA by hybridization;
wherein a detectable signal is generated upon binding of said chromophore to said CBR and upon interaction between said TBR and said target small RNA.

3. A molecular sensor for detecting a target molecule comprising:
a nucleic acid comprising:
a first chromophore binding region (CBR) comprising an aptamer sequence with specific binding affinity for a chromophore, said chromophore being endogenous in a cell; and
a target molecule binding region (TBR) comprising a nucleic acid sequence selected to interact with a target small RNA by hybridization;
wherein a detectable signal is generated upon binding of said chromophore to said CBR.

4. A molecular sensor for detecting a target molecule comprising:
a nucleic acid comprising:
a first chromophore binding region (CBR) comprising an aptamer sequence with specific binding affinity for a chromophore, said chromophore being endogenous in a cell; and
a target molecule binding region (TBR) comprising a nucleic acid sequence selected to interact with a target small RNA in said cell by hybridization;
wherein a detectable signal is generated upon binding of said chromophore to said CBR and upon interaction between said TBR and said target small RNA.

5. A molecular sensor for detecting a target molecule comprising:
a nucleic acid comprising:
a first chromophore binding region (CBR) comprising an aptamer sequence with specific binding affinity for a chromophore, said chromophore being biliverdin;
wherein a detectable signal is generated upon binding of said chromophore to said CBR.

6. A molecular sensor for detecting a target molecule comprising:
a nucleic acid comprising:
a first chromophore binding region (CBR) comprising an aptamer sequence with specific binding affinity for a chromophore, said chromophore being biliverdin; and
a target molecule binding region (TBR) comprising a nucleic acid sequence selected to interact with a target molecule;
wherein a detectable signal is generated upon binding of said chromophore to said CBR and upon interaction between said TBR and said target molecule.

7. A molecular sensor for detecting a target molecule comprising:
a nucleic acid comprising:
a first chromophore binding region (CBR) comprising an aptamer sequence with specific binding affinity for a chromophore, said chromophore being biliverdin.

8. The molecular sensor of claims 1-7, wherein said aptamer sequence is selected from the group consisting of SEQ IDs 1-185.

9. The molecular sensor of claims 1-6, wherein said detectable signal comprises a change in an optical property.

10. The molecular sensor of claims 1-6, wherein said detectable signal comprises a change in an optical property selected from the group consisting of a fluorescence change, an absorbance change, a luminescence change, and a phosphorescence change.

11. The molecular sensor of claims 1-6, wherein said first CBR is functionally coupled to said TBR to prevent binding of said chromophore in the absence of said target molecule.

12. The molecular sensor of claims 1-6, wherein said first CBR is functionally coupled to said TBR by a transducer stem forming region.

13. The molecular sensor of claims 1-7, further comprising a second CBR, wherein said second CBR generates a fluorescent signal upon binding of said chromophore to said first CBR.

14. The molecular sensor of claims 1-7, further comprising a nucleic acid sequence which interacts with a particular RNA-binding protein.

15. The molecular sensor of claims 1-7, further comprising a nucleic acid sequence which interacts with an RNA-binding protein selected from the group consisting of dCas9 and MS2.

16. The molecular sensor of claims 1-7, wherein said nucleic acid is selected from the group consisting of RNA, DNA, artificially modified nucleotide-containing nucleic acids and combinations thereof.

17. The molecular sensor of claims 1-7, wherein said nucleic acid is a single-stranded transcription or synthesis product.

18. The molecular sensor of claims 1-7, further comprising a protective feature attached to said nucleic acid.

19. The molecular sensor of claims 1-7, further comprising a protective feature attached to nucleic acid tag which hybridizes to at least a portion of said nucleic acid selected from the group consisting of a bead, an RNA-binding protein and a modified nucleotide.

20. A method for detecting target molecules in a cell comprising:
introducing an expression package into a cell, said expression package comprising:
a double-stranded deoxyribonucleic acid (DNA) incorporating an operative promoter coupled to a gene encoding a nucleic acid transcript comprising a first chromophore binding region (CBR) comprising an aptamer sequence with specific binding affinity for a chromophore which is endogenous to said cell and a target molecule binding region (TBR) comprising a nucleic acid sequence selected to interact with a target molecule, wherein a fluorescent signal is generated upon binding of said chromophore to said CBR and upon interaction between said TBR and said target molecule;
expressing said expression package in said cell; and
detecting said fluorescent signal to determine the presence of said target molecule in said cell.

21. A method for detecting target ribonucleic acid (RNA) molecules in a cell comprising:
introducing an expression package into a cell, said expression package comprising:
a double-stranded deoxyribonucleic acid (DNA) incorporating an operative promoter coupled to a gene encoding an RNA transcript comprising a first chromophore binding region (CBR) comprising an RNA-aptamer sequence with specific binding affinity for a chromophore and a target molecule binding region (TBR) comprising an RNA sequence selected to interact with a target RNA, wherein a fluorescent signal is generated upon binding of said chromophore to said CBR and upon interaction between said TBR and said target RNA;
expressing said expression package in said cell; and
detecting said fluorescent signal to determine the presence of said target RNA in said cell.

22. A method for selecting molecular sensor sequences comprising:
introducing an expression package into a cell containing an endogenous chromophore, said expression package comprising:
a double-stranded deoxyribonucleic acid (DNA) incorporating an operative promoter coupled to a gene encoding a nucleic acid transcript comprising a first chromophore binding region (CBR) comprising an aptamer sequence with specific binding affinity for said endogenous chromophore;
expressing said expression package in said cell;
interrogating said cell for a fluorescence signal generated upon said first CBR binding to said endogenous chromophore; and
sorting said cell if said fluorescence signal is detected.

23. The method of claims 20-22, wherein said first CBR has a specific binding affinity to biliverdin.

24. The method of claims 20-22, wherein said first CBR is a non-naturally occurring nucleic acid sequence which binds to biliverdin with specificity and having substantial homology or identity to a sequence selected from the group consisting of SEQ IDs 1-185.
